(19) United States
(12) Patent Application Publication
(10) Pub. No.: US 2013/0260433 A1
(43) Pub. Date: Oct. 3, 2013

(54) HIGH RATE ANAEROBIC DIGESTER SYSTEM AND METHOD

(71) Applicant: Ruihong Zhang, Davis, CA (US)

(72) Inventor: Ruihong Zhang, Davis, CA (US)

(73) Assignee: The Regents of the University of California, Oakland, CA (US)

(21) Appl. No.: 13/697,125

(22) PCT Filed: May 16, 2011
(86) PCT No.: PCT/US11/36697

§ 371 Date: Jan. 29, 2013

Related U.S. Application Data
(60) Provisional application No. 61/345,029, filed on May 14, 2010.

(51) Int. Cl. C12M 1/107

(52) U.S. Cl. CPC C12M 21/04
USPC 435/167; 435/290.4; 435/168; 435/170; 435/290.2

(57) ABSTRACT

An anaerobic digester system for producing a biogas from organic material is disclosed. The system includes a hydrolysis reactor comprising therein acidogenic and hydrolytic bacterial culture for which the organic material is a hydrolysis substrate, a biogasification reactor comprising therein acetogenic and methanogenic bacterial culture, and a biostabilization reactor comprising therein a methanogenic bacterial culture. The operating conditions of the biostabilization reactor are tailored to increase the digestion rate and energy conversion efficiency of the system. A method of using the system is also disclosed.

### CROSS-REFERENCE TO RELATED APPLICATIONS

This PCT Application claims priority to U.S. Provisional Patent Application No. 61/345,029, filed on May 14, 2010, the disclosure of which is incorporated herein by reference in its entirety for all purposes.

### BACKGROUND OF THE INVENTION

1. Field of the Invention

This invention relates, in general, to high rate anaerobic digester systems and methods for their use.

2. Description of Related Art

Biodigestion has long been known as a process for treatment of sewage, and for environmental protection. More recently biodigestion has gained prominence in the field of renewable energy generation. The biogas produced during the biodigestion process can be used to run generators for electricity production and boilers for heating purposes.

FIGS. 1A, 1B, and 1C illustrate conventional digester system designs making use of a single reactor vessel. FIG. 1A illustrates a dry anaerobic composting (Dranco) process. The Dranco digester is a dry, single-stage, thermophilic anaerobic digestion system (Verma, 2002). Feed is introduced into the top of the digester and flows to the conical bottom where an auger removes the digestate. A fraction of the digestate is used to inoculate incoming feed and steam is added to increase the temperature to thermophilic range. The rest of the digestate is dewatered to produce wastewater and press cake. The press cake contains active bacteria, some ammonia, and undigested solids that are aerobically stabilized for use as compost. Source-separated waste is preferred in order to maintain the quality of the compost. There is no mixing within the reactor, other than some bubbling of biogas against the downward plug-flow motion of the substrate. Dranco digesters were reported to have maintained a high average organic loading rate (12-15 kg VS/m³/d) for treating municipal organic solid wastes.

FIG. 1B illustrates a system for performing a Kompogas process. The Kompogas digester is a high-solids plug-flow design. The cylindrical reactor is oriented horizontally and contains internal rotors that assist in degassing and homogenization (Lissens et al. 2001; Nichols 2004). The system is prefabricated in 15,000 or 25,000 ton/year (t/y) sizes. The internal solids content has to be carefully maintained at 23-28% in order for the system to flow properly. Therefore, some process water and digestate is mixed with incoming organic waste, which also provides inoculation (Lissens et al., 2001). Retention time is 15-20 days under thermophilic conditions.

FIG. 1C illustrates a Valorga system. The Valorga digester is a dry, single-stage digester that treats organic solid waste with 25-30% total solids (TS) (Nichols, 2004). Unlike other plug-flow digesters, the Valorga design uses pressurized biogas for mixing. This eliminates the need for an inoculation loop. The vertical cylindrical digester contains a partition extending across two-thirds of the digester's diameter. This forces material entering at the bottom to flow around the wall before exiting (de Laclos et al., 1997). According to Nichols (2004), feedstocks with less than 20% total solids (TS) do not perform well in the Valorga system because grit particles settle and clog the biogas injection ports. The retention time is 21 days and the biogas yields are reported to be 220-270 m³/t VS (Nichols, 2004).

FIG. 2 illustrates a sequential batch anaerobic composting (SEBAC) system including two or three batch, leach-bed digesters loaded in sequence such that leachate can be transferred between digesters by a sprayer (Chynoweth et al., 1991; Chynoweth et al., 1992; Okeefe et al., 1993; Forster-Carneiro et al., 2004). Roughly-chopped organic fraction of municipal solid waste (OFMSW) is placed in a batch digester. Leachate from a mature digester is sprayed onto the fresh material as an inoculant, while leachate is recycled to the top of the mature pile until methanogenesis stabilizes. The digester is then switched over to internal recirculation until methane production slows as the batch matures. In laboratory trials the SEBAC process had difficulty starting when loaded with pure food waste (Forster-Carneiro et al., 2004). Bulking agents were required to prevent compaction and allow leachate to drain. An early pilot study reported methane yields of 160 and 190 m³/t VS when the retention time was 21 and 42 days, respectively (Chynoweth et al., 1992). The waste stream contained 60% paper and cardboard, 10% plastic, and 6% yard waste, and the authors reported that the yields represented 80-90% of the ultimate methane potential.

Conventional anaerobic phase solids (APS) systems break down biodegradable material to manage waste, produce a biogas, or both. Existing APS systems operate on the principle of anaerobic digestion of solids in staged phases.

Because anaerobic digestion uses a mixed and highly competitive microbial culture that can degrade essentially all the biodegradable components in organic matter, anaerobic digestion has been one of the key technologies for waste degradation and treatment. Compared to other biomass conversion technologies, such as ethanol fermentation, anaerobic digestion is less costly and more adaptable in different sizes for distributed operations. The bacteria and fungi used in anaerobic digestion processes possess effective enzyme systems to break down organic polymers, such as fibers (e.g. cellulose and hemicellulose), protein and fat. Over the years, anaerobic digestion technologies evolved from solely an environmental management process to a viable process for the production of renewable energy. With increasing demands for renewable energy and reducing greenhouse gas emissions and environmental degradation, anaerobic digestion has gained greater attention.

Exemplars of existing high solids anaerobic digesters are U.S. Pat. No. 6,342,378 to Zhang et al. and U.S. Pat. No. 7,556,737 to Zhang. These patents disclose anaerobic phased solids digesters (APS-Digester) developed at the University of California, Davis (UC Davis). The APS Digesters combine the features of batch and continuous digesters (Zhang and Zhang, 1999; Zhang, 2002; Hartman, 2004). The exemplary system includes five reactors: four hydrolysis reactors and one biogasification reactor. Feedstock is loaded into each of the hydrolysis reactors and acted on by extracellular enzymes and acidogenic bacteria that liquefy the waste and converts it to simple organic acids. The acids are collected and transferred to the biogasification reactor where they are reduced further to methane by methanogenic bacteria. Multiple hydrolysis reactors allow for a time separation between the beginnings of each batch hydrolysis reaction. This time separation contributes to a relatively consistent biogas production rate despite the batch loading and operational schedule. After each batch has been completely digested, the solids and liquid are removed and stabilized aerobically. In laboratory studies, the APS Digester system was able to digest rice straw reducing 40-60% of the TS and producing 400-500 m³ biogas/t VS, which is comparable to yields seen for more easily degradable substrates (Zhang and Zhang, 1999). Other substrates tested on the APS Digester include post-consumer food waste, food processing waste, and animal manure. Biogas yields from restaurant food waste and green waste (grass clippings) were 600 and 440 m³/t VS, respectively, with a 12 d retention time and biogas production rate of 3-3.5 m³/m³/d.

Loom Existing anaerobic digestion technologies are separated into two categories—those for treating solid waste and those for treating wastewater. For applications where both solid and liquid materials need to be treated, for example in a food processing plant, the existing technologies have limitations.

There is a continuing need to increase the performance of anaerobic digestion technologies. Existing anaerobic digesters require a significant incubation time for digestion and biostabilization of the contents before the effluent can be discharged. Additionally, there is a continuing need for efficiency improvements both in terms of increased yield and lower costs.

In light of the foregoing, it would be beneficial to have methods and apparatus which overcome the above and other disadvantages of known biodigesters including anaerobic digesters.

### SUMMARY OF THE INVENTION

Various aspects of the invention are directed to an anaerobic digester system for producing a biogas from organic material. The system includes a hydrolysis reactor comprising therein acidogenic and hydrolytic bacterial culture for which the organic material is a hydrolysis substrate, a biogasification reactor comprising therein acetogenic and methanogenic bacterial culture, and a biostabilization reactor comprising therein a methanogenic bacterial culture. The hydrolysis reactor further includes an inlet port for receiving the organic material, an outlet port for discharging hydrolysis effluent from the hydrolysis reactor, and a gas vent for discharging the biogas from the hydrolysis reactor. The biogasification reactor further includes a biogasification reactor inlet port for receiving the hydrolysis effluent from the hydrolysis reactor outlet port, a reactor outlet port for discharging biogasification effluent from the biogasification reactor, and a gas vent for discharging the biogas from the biogasification reactor. The biostabilization reactor further includes a biostabilization reactor inlet port for receiving the biogasification effluent from the biogasification reactor outlet port, a biostabilization reactor outlet port for discharging biostabilization effluent from the biostabilization reactor, and a gas vent for discharging the biogas from the biostabilization reactor.

In various embodiments, the biogasification reactor has a controlled internal pH of between about 6.8 and about 8.2. In various embodiments, the biostabilization reactor has a controlled internal pH of between about 6.8 and about 8.2. In various embodiments, the biogasification reactor has a controlled internal temperature between about 25° C. and about 55° C. In various embodiments, the biostabilization reactor has a controlled internal temperature equal to or below that of the biogasification reactor. In various embodiments, the biostabilization reactor has a controlled internal temperature between about 25° C. and about 55° C.

In various embodiments, the organic material is a member selected from a solid, liquid, and a combination thereof.

In various embodiments, the biostabilization reactor bacterial culture is essentially methanogenic. In various embodiments, the biostabilization reactor bacterial culture is essentially free of acetogenic bacteria.

In various embodiments, the system further includes a grinder upstream from the biogasification reactor for mechanically reducing the size of solid particles in the feedstock. In various embodiments, the system further includes a solid-liquid separator positioned between the biogasification reactor and the biostabilization reactor, the separator configured to separate fibrous solid components from a liquid component of the effluent from the biogasification reactor. In exemplary embodiments, the fibrous solid component has a moisture content between about 60% and about 75%. The exemplary grinder optionally grinds the materials from the hydrolysis reactor, and the ground material is sent back to the hydrolysis reactor. In the exemplary system, the grinder grinds the hydrolysis effluent before it is transferred back to the biogasification reactor.

Various aspects of the invention are directed to a biostabilization reactor system for producing a biogas from a partially-digested organic material. The biostabilization reactor system includes a vessel including an inlet for mixing the partially-digested organic material with a biostabilization bacterial culture for biodigestion of the organic material, a gas vent for discharging biogas resulting from the biogasification, and an outlet port for discharging liquid effluent resulting from the biogasification from the vessel. The partially-digested organic material has been submitted to methanogenesis with a mixture of acetogenic and methanogenic bacterial culture upstream from the vessel. The biostabilization bacterial culture is a methanogenic culture.

In various embodiments, the system further includes a solid-liquid separator for separating solid components from liquid components of the partially-digested organic material to be fed to the biostabilization vessel. In various embodiments, biostabilization reactor vessel is configured to maintain an internal temperature of between about 25° C. to about 55° C. The biostabilization reactor vessel may be configured to maintain the mixture of the organic material and the biostabilization bacterial culture at a pH of between about 6.8 and about 8.2.

In various embodiments, the biostabilization outlet port is configured to draw the liquid effluent from a region adjacent the inner wall surface of the biostabilization vessel. The discharged biogas may be discharged from a top of the biostabilization vessel.

In various embodiments, the method further includes recycling a portion of the separated liquid from the solid-liquid separator to the hydrolysis reactor. The effluent from one or more of the reactors may be transferred to one or more of the other reactors. In various embodiments, the biogasification effluent is recycled to the hydrolysis reactor. In various embodiments, the biostabilization effluent is recycled to the hydrolysis reactor. In various embodiments, the biostabilization effluent is recycled to the biogasification reactor. In various embodiments, the effluent from a reactor is recycled back into the respective reactor. The recycled effluent may be a liquid, solid, or combination thereof. In various embodiments, the recycled effluent is a liquid, and the effluent is added to the feedstock for the hydrolysis reactor to adjust the moisture content thereof. The liquid may be processed to remove ammonia and other constituents (e.g. salt elements) prior to recycling to the hydrolysis reactor.

Various aspects of the invention are directed to a method of producing a biogas from organic material. The method includes delivering a feedstock to the hydrolysis reactor of the system, incubating a hydrolysis mixture comprising the hydrolysis effluent and the acidogenic and hydrolytic bacterial culture under anaerobic conditions to produce hydrogen, carbon dioxide, and the hydrolysis effluent, transferring at least a portion of the hydrolysis effluent to the biogasification reactor, incubating a biogasification mixture comprising the hydrolysis effluent and the acetogenic and methanogenic bacterial culture under anaerobic conditions to produce methane, carbon dioxide, and the biogasification effluent, transferring at least a portion of the biogasification effluent to the biostabilization reactor, and incubating a biostabilization mixture comprising the biogasification effluent and the biostabilization methanogenic bacterial culture under anaerobic conditions to produce methane and the biostabilization effluent.

In various embodiments, each of the steps is performed essentially simultaneously.

The system and method of the present invention(s) have other features and advantages which will be apparent from or are set forth in more detail in the accompanying drawings, which are incorporated in and form a part of this specification, and the following Detailed Description of the Invention, which together serve to explain the principles of the present invention(s).

### BRIEF DESCRIPTION OF THE DRAWINGS

FIGS. 1A, 1B, and 1C are schematic views of conventional biodigesters.

FIG. 2 is a schematic view of a conventional sequential batch anaerobic composting (SEBAC) system.

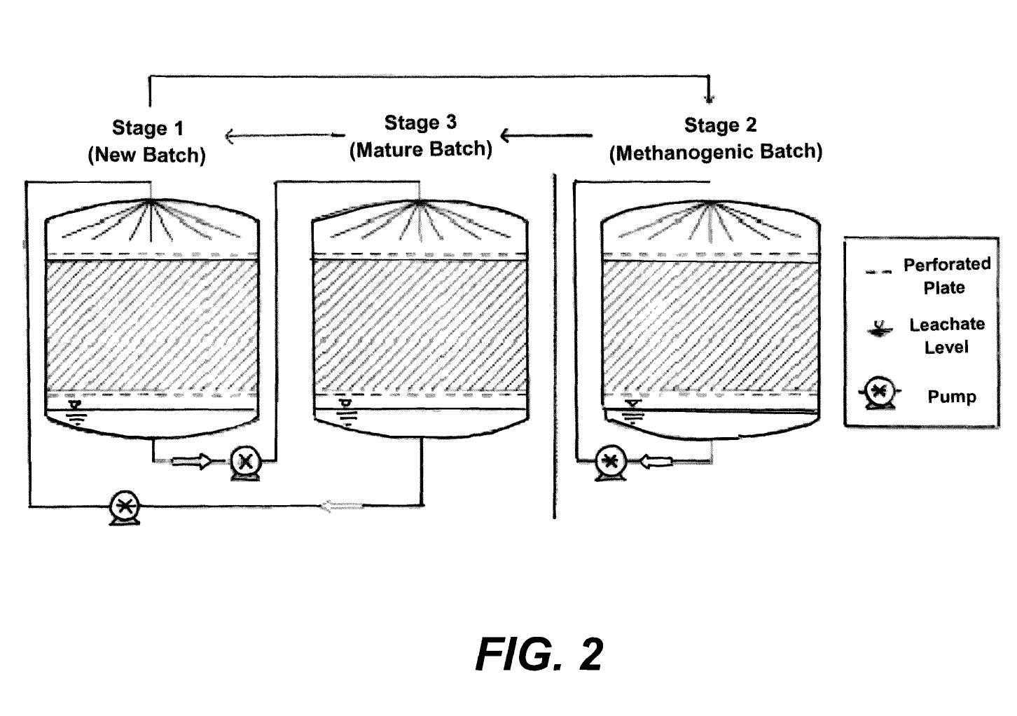

FIG. 3 is a schematic view of the biochemical processes involved in the anaerobic digestion process of the present invention.

FIG. 4 is a schematic view of the anaerobic digester system in accordance with the present invention.

FIG. 5 is a schematic view of an anaerobic digester system similar to the system of FIG. 4, illustrating use of the optional ammonia removal device.

FIG. 6 is a schematic view of an anaerobic digester system similar to the system of FIG. 4, illustrating use of the optional ammonia removal device and addition of a fresh liquid feed to the biogasification reactor.

FIG. 7 is a schematic view of an anaerobic digester system of the invention in which hydrolysis reactor effluent is transferred directly into the biostabilization reactor.

FIG. 8 is a schematic view of an anaerobic digester system of the invention in which effluent from the biostabilization reactor is recycled into the digester system, e.g., through the hydrolysis reactore via valve 37.

FIG. 9 is an illustration of the public benefits of biogas products produced in accordance with the system and method of the present invention.

### DETAILED DESCRIPTION OF THE INVENTION

Reference will now be made in detail to the various embodiments of the invention, examples of which are illustrated in the accompanying drawings. While the invention will be described in conjunction with the various embodiments, it will be understood that they are not intended to limit the invention to those embodiments. On the contrary, the invention is intended to cover alternatives, modifications and equivalents, which may be included within the spirit and scope of the invention as defined by the appended claims

Definitions and Abbreviations

“Biogas” refers to a gas produced by the biological breakdown of organic matter, typically in the absence of oxygen. Examples of biogas include, but are not limited to, methane, hydrogen, and carbon dioxide produced by anaerobic digestion, fermentation, or biogasification of biodegradable materials such as biomass, manure, sewage, municipal waste, green waste and crops.

“Biogasification” refers generally to the production of a biogas product by microorganisms from an organic material. In various aspects “biogasification” refers to the production of biogas in the treatment of a liquid or solid feedstock or material of the present invention. In various respects, “biogasification” refers to the process by which methane and/or carbon dioxide are produced from an organic material by the process and system of the present invention.

“Anaerobic digestion” is to be understood as is generally used in the industrial, chemical, agricultural, and environmental arts. In various aspects, “anaerobic digestion” refers to a series of processes by which microorganisms break down organic or biodegradable material, in the absence of oxygen, to manage waste and/or to release energy. In various aspects, “anaerobic digestion” refers to the processing of various organic material including liquid, solid, and combinations thereof. “Anaerobic digestion” is used interchangeably with “AD” and “digestion”.

“Methanogenesis” and “biomethanation” are used interchangeably and refer to the formation of methane by methanogens. In various aspects, “methanogenesis” is used interchangeably with “biogasification.” “Methanogenesis” is to be understood as generally used in the industrial, chemical, agricultural, and environmental arts and refers in general to the formation of methane by microorganisms known as methanogens. In various respects, methanogenesis occurs by anaerobic fermentation.

“Methanogens” and “methanogenic bacterial culture” are to be understood as generally used in the environmental, agricultural, and chemical arts and refers broadly to the category of microorganisms capable of producing methane from organic material and/or metabolizing organic material. Exemplary methanogens include, but are not limited to, Methanobacterium oinelianskii, Mb. formicium, Mb. sohngenii, Methanosarcina barkeri, Ms. methanica and Mc. mazei, and combinations thereof. Also of use are Methanobacteriaceae, Methanosarcinaceae, Methanosaetaceae, Methanocorpusculaceae, Methanomicrobiaceae, and other archae organisms.

“Acetogens” and “acetogenic bacterial culture” are to be understood as generally used in the environmental, agricultural, and chemical arts and refer broadly to a category of microorganisms capable of producing acetate as a product of anaerobic fermentation.

“Acidogens” and “acidogenic bacterial culture” are to be understood as generally used in the environmental, agricultural, and chemical arts and refer broadly to the category of microorganisms capable of producing volatile fatty acids as a product of anaerobic fermentation.

“Bacterial culture” is to be understood as generally used in the agricultural, chemical, and environmental arts. In various respects, “bacterial culture” refers to a mixed culture. In various respects, “bacterial culture” includes bacteria and archaea.

“Organic substrate”, “organic material”, and “feedstock” are used essentially interchangeably and refer to material that can be used in the process and system of the invention to produce a biogas product. In some respects, “organic substrate” refers to carbonaceous material that can be used in the process and system of the present invention. “Organic substrate” may refer to a liquid, solid, or combination of the same.

In an exemplary embodiment, the organic substrate is food waste and municipal solid waste. Previous research has demonstrated the feasibility of anaerobically digesting food waste and its mixture with agricultural waste, such as animal manure and municipal solid waste (Zhang et al., 2006; El-Mashad and Zhang, 2010; Zhu et al., 2010). In various respects, the organic material may be pretreated by a chemical treatment such as acid treatment, alkaline treatment, radiation treatment, heat treatment, radiation treatment, ammonia treatment, and combinations thereof.

“Organic material” and “feedstock” are used essentially interchangeably. “Feedstock” is to be understood as used in the agricultural and environmental arts.

“Partially-digested” refers to an organic material that has been subjected to a biogasification process. In various respects, “partially-digested” refers to an organic material in which at least a significant part has been subjected to hydrolysis or in which at least a significant part has been subjected to acetogenic and methanogenic bacterial culture.

“Hydrolysis” is to be understood as generally used in the industrial, chemical, agricultural, and environmental arts. “Hydrolysis” generally refers to the splitting of a molecule into two or more parts by the addition of a molecule of water. In various respects, “hydrolysis” refers to the chemical reaction by which molecules of water are split into hydrogen cations and hydroxide anions. In various respects, “hydrolysis” refers to process by which hydrogen and/or carbon dioxide are produced from an organic material by the process and system of the present invention. In various respects, “hydrolysis” refers to the process by which hydrogen is produced by the metabolizing of an organic material by hydrolytic microorganisms.

“Hydrolytic microorganisms” is to be understood as generally used in the industrial, chemical, agricultural, and environmental arts and refers broadly to a category of microorganisms capable of producing hydrogen as a product of anaerobic respiration. Exemplary hydrolytic microorganisms include, but are not limited to, Clostridium, Lactobacillus and other Firmicutes and Proteobacteria, and combinations thereof.

“Solid-liquid separator” refers generally to a device for separating solid components from liquid components in accordance with the process and system of the invention. In various respects, “solid-liquid separator” refers to a device for increasing the amount of separation between the solid components and liquid components from a level that occurs in the absence of the device. In various respects, “solid-liquid separator” refers to a device for separating solid particles having a diameter larger than about 1 mm, less than about 3 mm, less than about 5 mm, less than about 10 mm, or less than about 20 mm.

“Reactor”, “vessel”, and “reactor vessel” are essentially used interchangeably to refer to a device in which a material is housed, and in some respects, a device in which a reaction according to the present invention occurs.

“Incubation” is to be understood as generally used in the chemical, agricultural, and environmental arts. As used herein, “incubation” refers generally to allowing a material to sit for a period of time for a desired action to occur.

As used herein, “fluid” refers broadly to a liquid, with or without suspended solid material. In various respects, the solids are in an amount that allows the “fluid” to be flowable through the system of the invention.

“Solid” is to be understood as generally used in the chemical, agricultural, and environmental arts. “Solid” includes, but is not limited to, an inert solid, soluble solid, biodegradeadable solid, and non-biodegradeadable solid. In various respects, “solid” refers to a biodegradable solid.

“Acid”, “base”, and “salt” are to be to be understood as these terms are generally used in the chemical, agricultural, and environmental arts.

“HR” refers to the “hydrolysis reactor”. “BGR” refers to the “biogasification reactor”. “BSR” refers to the “biostabilization reactor”.

For convenience in explanation and accurate definition in the appended claims, the terms “up” or “upper”, “down” or “lower”, “inside” and “outside”, and “top” and “bottom” are used to describe features of the present invention with reference to the positions of such features as displayed in the figures.

In many respects the modifications of the various figures resemble those of preceding modifications and the same reference numerals followed by subscripts “a”, “b”, “c”, “d”, and apostrophes designate corresponding parts.

Unless otherwise noted, the terms and abbreviations used herein are to be understood as generally used in the industrial, chemical, agricultural, and environmental arts. Unless otherwise noted, the use of the singular includes the plural and vice versa.

Various aspects of the invention are related to the digester systems and methods disclosed by U.S. Pat. No. 6,342,378 issued Jan. 29, 2002 and entitled BIOGASIFICATION OF SOLID WASTE WITH AN ANAEROBIC SOLIDS DIGESTER SYSTEM and U.S. Pat. No. 7,556,737 issued Jul. 7, 2009 and entitled ANAEROBIC PHASED SOLIDS DIGESTER FOR BIOGAS PRODUCTION FROM ORGANIC SOLIDS WASTE, the entire contents of which are incorporated herein for all purposes by this reference. By contrast to existing anaerobic digester systems, the system in accordance with the present invention achieves a higher process rate and higher energy conversion efficiency.

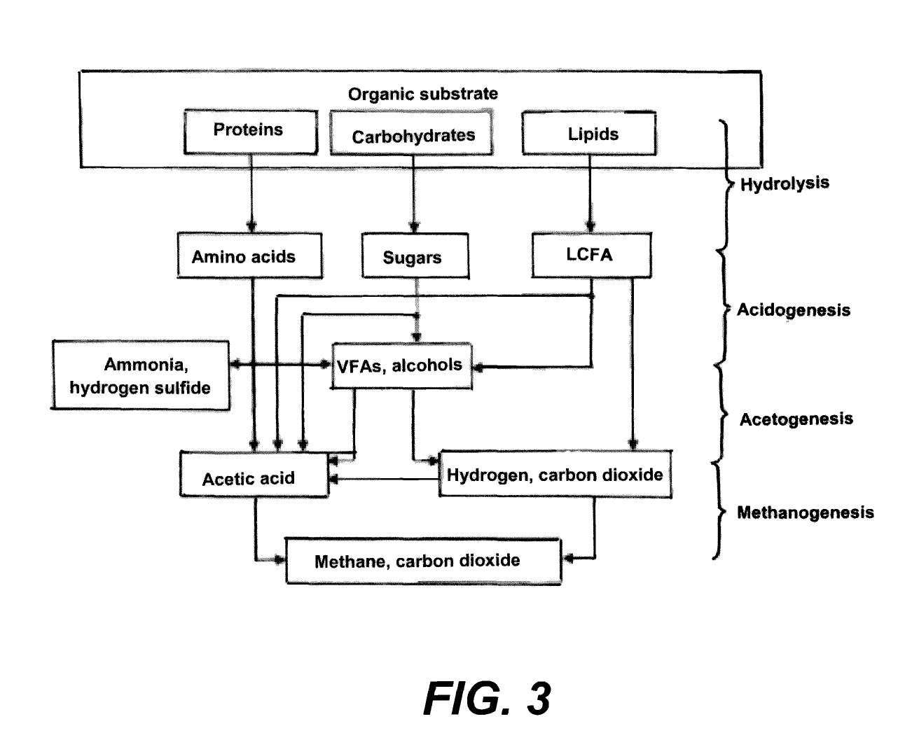

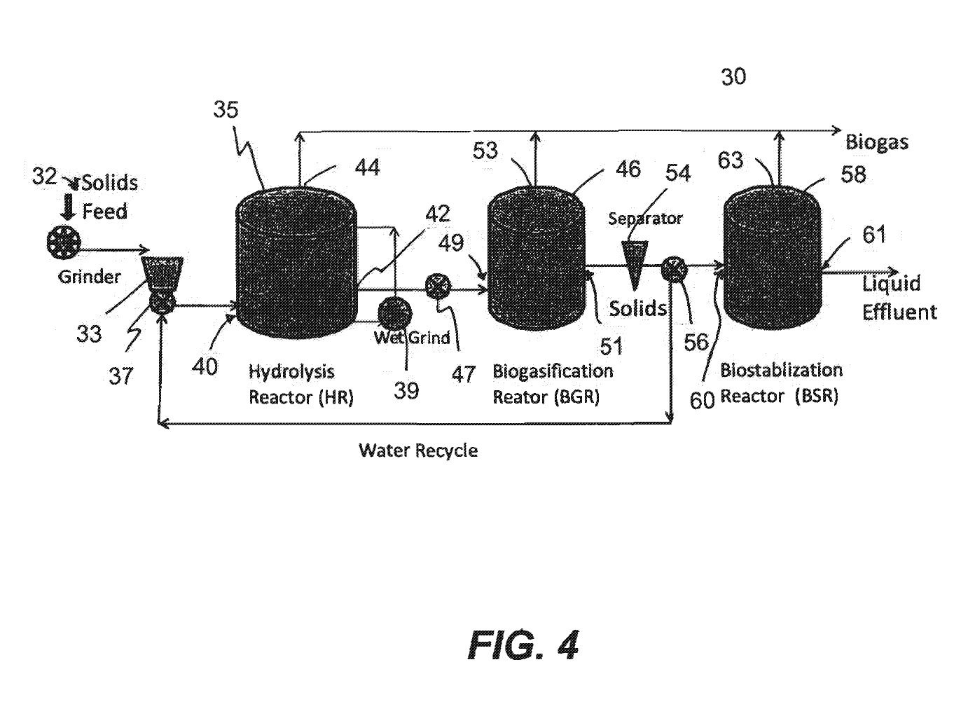

Turning now to the drawings, wherein like components are designated by like reference numerals throughout the various figures, attention is directed to FIGS. 3 and 4. FIG. 3 is a generalized illustration of the biochemical processes involved in the anaerobic digestion process. As shown in FIG. 3, during anaerobic digestion, the organic matter is hydrolyzed by extracellular enzymes of microorganisms to soluble compounds such as amino acids, sugars and long-chain fatty acids. Next the products of the hydrolysis step are fermented into short-chain volatile fatty acids (VFAs), alcohols, ammonia and hydrogen sulfide. The VFAs (other than acetate) and alcohols are further converted by acetogenesis bacteria to acetic acid, hydrogen, and carbon dioxide, which are then converted by methanogenic bacteria to methane and carbon dioxide. The biogas resulting from anaerobic digestion may contain hydrogen, methane, carbon dioxide as the main components and can be used as a fuel for electricity, heat generation, or fuel for transportation vehicles.

In an exemplary embodiment, the organic material or feedstock is agricultural waste, e.g., rice straw. Previous research has demonstrated the feasibility of anaerobically digesting a mixture of straw (rice straw and wheat straw) and other agricultural and food wastes, such as animal manure, green leaves and molasses, using conventional digestion reactors fed in batches or semicontinuously (Hills, D. J. and D. W. Roberts, Agricultural Wastes 3:179-189 (1981); Dar, G. H. and S. M. Tandon, Biological Wastes 21:75-83 (1987); Adbullah et al., Journal of Agricultural Sciences 119:255-263 (1992); Somayaji, D. and S. Khanna, World Journal of Microbiology & Biotechnology 10:521-523 (1994)). The research of Hills and Roberts (1981) showed that adding either chopped rice straw or chopped wheat straw to dairy manure enhanced the anaerobic digestion process and increased the methane production. Rice straw is a ligno-cellulosic material mainly composed of cellulose (37.4%), hemicellulose (44.9%), lignin (4.9%), and silicon ash (13.1%) (Hills, D. J. and D. W. Roberts, Agricultural Wastes 3:179-189 (1981)). The straw contains about 0.4% nitrogen and has a carbon to nitrogen ratio (C/N) of around 75. The proper range of C/N ratio for anaerobic digestion is 25-35 (Hills, D. J. and D. W. Roberts, Agricultural Wastes 3:179-189 (1981)). Therefore, nitrogen may need to be supplemented in order to effect the anaerobic digestion of rice straw.

Nitrogen can be added in inorganic forms, such as ammonia, or in organic forms such as organic nitrogen contained in urea, animal manure or food wastes. Once nitrogen is released from the organic matter, however, it will become ammonia (NH⁴⁺) which is water soluble. Recycling of nitrogen in the digested liquid will reduce the amount of nitrogen needed for continuous operation of anaerobic digesters. Animal manures and food wastes are good nutrient sources if they are readily available in the areas close to rice straw production. Nitrogen fertilizer, such as ammonia or urea, is another source of nitrogen that can be easily added to the straw and may be more suitable for the areas where handling other types of wastes is not feasible. Thus, in various embodiments, the organic material is supplemented with a nitrogen source. In various embodiments, the nitrogen source is a member selected from the group consisting of urea, animal manure, food waste, inorganic nitrogen fertilizers and combinations thereof.

In various embodiments, the organic material, particularly agricultural waste (e.g., rice straw) is pretreated by a chemical treatment method selected from the group consisting of bicarbonate treatment, alkaline peroxide treatment, radiation treatment, ammonia treatment and combinations thereof.

In various embodiments, the organic material is a solid, liquid, or combination of the same. The exemplary system processes both a solid material and a liquid material such as wasterwater.

FIG. 4 illustrates an anaerobic digester system, generally designated 30, for producing a biogas from organic material or feedstock. In the exemplary system, a solid feedstock 32 is fed to a grinder 33 for reducing the size of the solid particles.

The ground feedstock is fed to a hydrolysis vessel (hydrolysis reactor) 35 by a pump 37. The exemplary system optionally includes a wet grinder 39 for further continuously reducing the particle size of the solid components in the reactor.

The exemplary hydrolysis reactor includes an inlet port 40 for receiving the feedstock from the pump and a first outlet port 42 for discharging hydrolysis effluent. A gas vent 44 allows for discharge of biogas from hydrolysis vessel 35. The inlet port of any of the reactor vessels may be configured to receive a solid, liquid, or combination thereof.

Hydrolysis effluent from hydrolysis reactor 35 is fed to a biogasification reactor 46 via a biogasification pump 47. The biogasification reactor includes a biogasification bacterial culture for producing a biogas from an organic material that includes the hydrolysis effluent. The organic material is fed through an inlet 49 and exits through an outlet 51 for discharging biogasification effluent from the biogasification reactor. A BGR gas vent 53 for discharging biogas product is provided on the biogasification reactor.

Effluent from biogasification reactor 46 is optionally fed through a solid-liquid separator 54. The exemplary separator is a conventional device for separating particles of a desired size from the mixed organic material. The exemplary separator is provided in-line between the biogasification reactor and biostabilization reactor.

Downstream from solid-liquid separator 54, a BSR pump 56 transfers the organic material (biogasification effluent) to a biostabilization reactor 58. The biostabilization reactor includes a first inlet port 60 for receiving effluent from the biogasification reactor outlet port and an outlet port 61 for discharging biostabilization effluent from the biostabilization reactor. A gas vent 63 discharges biogas product from the biostabilization reactor.

The components of system 30 will now be described in more detail.

In various embodiments, pump 37 is a chopper pump. During the incubation period, the mixed contents of the hydrolysis reactor 35 are pumped through the chopper pump to reduce the particle size of the solid components of the feedstock. By reducing the size of the particles, the energy conversion efficiency of system 30 generally, and hydrolysis reactor 35 in particular, may be increased. As will be described below, system 30 also accommodates the addition of liquid feedstock.

Hydrolysis reactor 35 is configured to house a mixture or solution. In various embodiments, hydrolysis reactor vessel 35 includes a compartment with one or more internal vertical dividers similar to the vessels of the '378 patent. The exemplary reactor vessel includes a single, undivided compartment. The exemplary vessel is a closed compartment configured to house the ground feedstock material in an oxygen-free environment. The exemplary hydrolysis reactor is a standard cylindrical vessel without a stirrer, auger, or other mixing devices. One will appreciate from the description herein, however, that the vessel may be provided with mixing or agitating devices to be used during the process of adding or removing organic material, or during part or all of the incubation period. Such devices include, but are not limited to, overhead stirrers, gas or motor driven stirrers, magnetic stirrers, shakers, homogenizers, sonicators, gas bubbling tubes, and ebulliators.

In various embodiments, hydrolysis outlet port 42 is in fluid communication with any interior surface of the hydrolysis reactor vessel. In the exemplary reactor, the outlet port is in fluid communication with a vertical surface of the hydrolysis reactor vessel. The outlet port may be connected directly, or indirectly, in known manner to a sidewall of the vessel to minimize drawing in of undesirable materials which typically collect in the central region of the vessel. In various embodiments, biogas is discharged from an interior of the reactor vessel. In the exemplary system, the gas vent is connected to a top of the vessel. In various embodiments, the gas vent is connected to a top portion of the vessel above a surface of the liquid contents.

The exemplary hydrolysis reactor contains a slurry or mixture of acidogenic and hydrolytic bacterial culture. The bacterial culture may be mixed with an aqueous base such as water. In the exemplary system, the bacterial culture is introduced into the hydrolysis reactor via the inlet port. The hydrolysis bacterial culture acts as a hydrolysis substrate for the organic feedstock material during incubation.

During operation, the hydrolysis bacterial culture and feedstock are mixed inside the hydrolysis reactor. In various embodiments, the hydrolysis vessel contains a mixture of organic feedstock, bacterial culture, and aqueous liquid equal to at least 50%, preferably at least 60%, more preferably at least 70%, even more preferably at least 80% and even more preferably at least 90%, 95% or essentially 100% of the internal capacity of the hydrolysis vessel.

In various embodiments, the internal temperature of hydrolysis reactor 35 during incubation is maintained in the range of about 25° C. to about 55° C., and preferably in the range of about 50° C. to about 55° C. In various embodiments, the internal pH of the feedstock and bacterial culture in hydrolysis reactor 35 during incubation is between about 4.0 to about 7.0. In various embodiments, chemicals are added to adjust the pH. The chemicals may be added through an inlet port or by other known methods.

The mixture of feedstock and hydrolytic bacterial culture is hydrolyzed to soluble compounds such as amino acids, sugars and long-chain fatty acids by extracellular enzymes of microorganisms. One will appreciate that any active hydrolytic- producing mesophilic or thermophilic organisms can be used for the hydrolytic bacterial culture. The hydrolytic bacterial culture may include, but is not limited to, microorganisms from the Clostridium species, Lactobacillus species, and Eubacteria species. The Clostridium species includes, but is not limited to, C. thermolacticum, C. thermohydrosulfuricum, C. thermosuccinogene, C. butyricum, C. pasteurianum, and C. beijirincki. The Lactobacillus species includes, but is not limited to, a Lactobacillus paracasel. The Eubacteria species includes, but is not limited to, an E. aerogenes. Other useful microorganisms and mixtures of microorganisms for use in hydrolysis reactor 35 will be apparent to those of skill in the art from the description herein.

An exemplary operative mixed culture of microorganisms is capable of sustaining itself indefinitely as long as a fresh supply of organic materials is added because the major products of the fermentation process are gases, which escape from the medium leaving little, if any, toxic growth inhibiting products. Mixed cultures generally provide the most complete fermentation action. Nutritional balance and pH adjustments can be made as will be appreciated from the description herein to favor hydrolytic activity.

Biogasification reactor 46 is physically configured similar to hydrolysis reactor 35. In various embodiments, biogasification outlet port 51 is in fluid communication with any interior surface of the biogasification reactor vessel. In the exemplary reactor, the outlet port is in fluid communication with a vertical surface of the biogasification reactor vessel. The outlet port may be connected directly, or indirectly, in known manner to a sidewall of the vessel to minimize drawing in of undesirable materials which typically collect in the central region of the vessel. In various embodiments, biogas is discharged from an interior of the reactor vessel. In the exemplary system, the gas vent is connected to a top of the vessel. In various embodiments, the gas vent is connected to a top portion of the vessel above a surface of the liquid contents.

In various embodiments, biogasification reactor 46 is configured to process a member selected from a liquid, solid, and combination thereof. In the exemplary system, wet grinder 39 reduces the size of the solid particles in the hydrolysis effluent from hydrolysis reactor 35. In an exemplary embodiment, the organic material retains small solid components for processing in the biogasification reactor. Any solids remaining in the organic material after processing in the biogasification reactor are optionally removed downstream by solid-liquid separator 54.

The exemplary biogasification reactor contains an acetogenic and methanogenic bacterial culture generally referred to as a biogasification bacterial culture. In various embodiments, the biogasification bacterial culture includes one of acidogens, acetogens, methanogens, and a combination thereof in varying amounts. The bacterial culture may be mixed with an aqueous base such as water by known processes. In various embodiments, the contents of one or more of the reactors is mixed with a mixing device. Exemplary mixing devices include, but are not limited to, impellers, stirrers, bubbling, and thermal cycling. During operation, the biogasification bacterial culture and effluent are mixed inside the biogasification reactor. In various embodiments, the biogasification vessel contains a mixture of organic material (effluent), bacterial culture, and aqueous liquid equal to at least 50%, preferably at least 60%, more preferably at least 70%, even more preferably at least 80% and even more preferably at least 90%, 95% or essentially 100% of the internal capacity of the biogasification vessel.

Methane-producing anaerobic systems utilizing acid-forming bacteria and methane-producing organisms, generally referred to as methanogens, may be employed to produce methane. A review of the microbiology of anaerobic digestion is set forth in Anaerobic Digestion, The Microbiology of Anaerobic Digestion, D. F. Toerien and W. H. J. Hattingh, Water Research, Vol. 3, pages 385-416, Pergamon Press (1969), which is incorporated herein for all purposes by this reference. As set forth in the Toerien review, the acid-forming species may include species from genera including, but not limited to, Aerobacter, Aeromonas, Alcaligenes, Bacillus, Bacteroides, Clostridium, Escherichia, Klebsiella, Leptospira, Micrococcus, Neisseria, Paracolobacterium, Proteus, Pseudomonas, Rhodopseudomonas, Sarcina, Serratia, Streptococcus and Streptomyces. Also of use in the present invention are microorganisms which are selected from the group consisting of Methanobacterium oinelianskii, Mb. formicium, Mb. sohngenii, Methanosarcina barkeri, Ms. methanica and Mc. mazei, and combinations thereof. Also of use are Methanobacteriaceae, Methanosarcinaceae, Methanosaetaceae, Methanocorpusculaceae, Methanomicrobiaceae, and other archae organisms.

A wide variety of substrates are utilized by the methane-producing bacteria, but each species is believed to be characteristically limited to the use of a few compounds. It is therefore believed that several species of methane producing bacteria are required for complete fermentation of the compounds present in certain organic substrates such as sewage. For example, the complete fermentation of valeric acid requires as many as three species of methane producing bacteria. Valeric acid is oxidized by Mb. Suboxydans to acetic and propionic acids, which are not attacked further by this organism. A second species, such as Mb. Propionicum, can convert the propionic acid to acetic acid, carbon dioxide and methane. A third species, such as Methanosarcina methanica, is required to ferment acetic acid.

The internal environment of the exemplary biogasification reactor is controlled to promote methanogenesis. In various embodiments, the internal temperature of biogasification reactor 46 is maintained above about 30° C. In various embodiments, the internal temperature of the biogasification reactor is maintained between about 25° C. and about 55° C. In various embodiments, the biogasification reactor has a controlled internal pH of between about 6.8 and about 8.2. In various embodiments, chemicals are added to adjust the pH.

Biostabilization reactor inlet 60 is in fluid communication with biogasification outlet 51. In various respects, biostabilization reactor 58 is configured similarly to biogasification reactor 46. Biostabilization reactor 58 includes a vessel for holding a mixture of organic material (e.g. effluent) and bacterial culture in an oxygen-free environment. The exemplary vessel is cylindrical. One will appreciate, however, that the vessel may have other shapes and configurations in accordance with the present invention similar to the hydrolysis reactor and biogasification reactor.

In various embodiments, one or more of the reactors includes a solid support for the bacterial culture such as a sheet, a plastic pellet, sand, a biofilm, and the like. The solid support promotes bacterial retention and increases bacterial population. Other substances such as silica can also be added to the reactors to promote the chemical and biochemical reactions therein.

In various embodiments, biostabilization outlet port 61 is in fluid communication with any interior surface of the biostabilization reactor vessel. In the exemplary reactor, the outlet port is in fluid communication with a vertical surface of the biostabilization reactor vessel. The outlet port may be connected directly, or indirectly, in known manner to a sidewall of the vessel to minimize drawing in of undesirable materials which typically collect in the central region of the vessel. In various embodiments, biogas is discharged from an interior of the reactor vessel. In the exemplary system, the gas vent is connected to a top of the vessel. In various embodiments, the gas vent is connected to a top portion of the vessel above a surface of the liquid contents.

The exemplary biostabilization reactor does not include a mixing device. One will appreciate that known mixing devices may be provided to for mixing and agitating including, but not limited to a stirrer or auger. In various embodiments, the biostabilization outlet port is in fluid communication with a vertical surface of the biostabilization reactor vessel. The outlet port may be connected directly, or indirectly, in known manner to a sidewall of the vessel to minimize drawing in of undesirable materials which typically collect in the central region of the vessel. In various embodiments, the biostabilization gas vent is connected to a top of the vessel. In various embodiments, the gas vent is connected to a top portion above a surface of the liquid contents.

In various embodiments, biostabilization reactor 58 is configured to process a member selected from a liquid, solid, and combination thereof. In various embodiments, biostabilization reactor is configured to process organic material which is essentially a liquid, meaning a liquid with no solids or only small, insignificant solid particles. In the exemplary system, optional solid-liquid separator 54 separates relatively large particles from the biogasification effluent before it is fed to the biostabilization reactor. In this manner, the biostabilization reactor can efficiently operate on the liquid components while solid components are primarily treated in hydrolysis reactor 35 and biogasification reactor 46. In various embodiments, the separated solid particles are added to the hydrolysis reactor feedstock. In various embodiments, the separated solid particles are processed off-line such as in a separate composting system.

In various embodiments, the biostabilization reactor is fed with a partially-digested organic material. The exemplary biostabilization reactor receives the partially-digested biogasification effluent. One of skill in the art will appreciate from the description herein how to adjust the level of processing by the biogasification reactor before transfer to the biostabilization reactor. The level of process is largely dependent on the composition of the organic material fed to the system. In the exemplary case of food waste serving as the feedstock, the hydrolysis effluent may be incubated in the biogasification reactor for a sufficient time to essentially use up all the solid components. The solid components in a straw feedstock, by contrast, can not easily be digested. In various embodiments, the amount of solid components digested in the biogasification reactor before transfer to the biostabilization reactor is about 70%, more preferably 75%, more preferably 80%, more preferably 85%, more preferably 90%, and more preferably 95%. In various embodiments, the organic material (hydrolysis effluent) is incubated in the biogasification reactor until essentially all of the solid components are digested.

As will be understood from the description herein, the minimum size of the particles to be separated from the biogasification reactor will depend on the application and system conditions. In various embodiments, the solid particles fed to biostabilization reactor 58 are greater than or equal to about 20 mm in diameter, preferably about 10 mm in diameter, and more preferably about 1 mm in diameter.

Unlike biogasification reactor 46, exemplary biostabilization reactor 58 contains methanogenic bacterial culture but is essentially free of acetogenic bacterial culture. Because the organic material (e.g. effluent) for the biostabilization reactor has been submitted to the exemplary biogasification reactor, which includes acetogenic and methanogenic bacterial culture, the organic material is partially digested before entering the biostabilization reactor. In various embodiments, the solid components are used up by the acetogenic bacterial culture in the biogasification reactor such that the biostabilization reactor can be customized for maximum energy conversion efficiency of the soluble components.

In various embodiments, the methanogenic bacterial culture in the biostabilization reactor 58 is essentially free of acetogenic bacteria. By “essentially free of acetogenic bacteria” it is meant that the culture contains minimal acetogenic bacteria and insignificant acidification reactions. In various respects, “essentially free of acetogenic bacteria” means hydrolyzable components would be reacted to an insignificant amount or not at all. In various respects, “essentially free of acetogenic bacteria” means less than about 10%, more preferably less than about 5%, more preferably less than 3%, and more preferably less than 1%. In various embodiments, the bacterial culture in the biostabilization reactor includes one of acidogens, acetogens, methanogens, and a combination thereof in varying amounts. In various embodiments, the bacterial culture in the biostabilization reactor includes one of acetogens, methanogens, and a combination thereof in varying amounts.

Any undigested solid components are separated by optional solid-liquid separator 54 and composted. The separated components may optionally be transferred back into biogasification reactor 46. In various embodiments, the solid components in the biogasification effluent are fibrous solids. In various embodiments, the moisture content of solid components in the biogasification effluent is between about 60% and about 75%. In various embodiments, the solid-liquid separator is a filter. In various embodiments, the solid-liquid separator is one of a grinder, grid, filter, sieve, strainer, slats, and combinations thereof for modifying the solid particle size, separating solid particles, or both in conventional manner. In certain exemplary embodiments, a strainer is used.

Because the solids components are submitted to the biogasification reactor and thereafter separated before the biostabilization reactor, neither the exemplary biogasification reactor nor the exemplary biostabilization reactor require a strainer or similar device in the respective inlets for preventing solids from entering. By contrast, existing anaerobic digestion systems include a single reactor vessel for methanogenesis and thus require a strainer or configuring the biogasification reactor to effectively use up the solid and liquid components, such as by extending the incubation time or increasing the vessel size.

In the exemplary system, solid components are digested or removed prior to being fed to the biostabilization reactor. Accordingly, the exemplary biostabilization reactor may be configured similar to a biomass retaining reactor. Exemplars of such biomass retaining reactors are biofilm reactors, upflow sludget blacket reactors, and anaerobic sequencing batch reactors. Reactors of the biomass retaining reactor type generally are used for processing wastewater and other organic material without solid components.

In various embodiments, the biostabilization vessel contains a mixture of organic material, bacterial culture, and aqueous liquid equal to at least 50%, preferably at least 60%, more preferably at least 70%, even more preferably at least 80% and even more preferably at least 90%, 95% or essentially 100% of the internal capacity of the biostabilization vessel. In various embodiments, the hydrolysis vessel, biogasification vessel, biostabilization vessel, or a combination thereof includes an empty volume of headspace above the solid and liquid contents for safety. In various embodiments, the headspace is equal to about 5% of the vessel volume.

In various embodiments, the internal temperature of biostabilization reactor 58 is maintained between about 25° C. to about 55° C., and preferably between about 25° C. to about 30° C. In various embodiments, the internal temperature of biostabilization reactor 58 is below the internal temperature of biogasification reactor 46. One will appreciate that the actual temperature inside the reactor vessels may fluctuate in reality and internal temperature may thus refer to an average temperature or temperature range. In various embodiments, biostabilization reactor 58 has an internal pH between about 6.8 and about 8.2 during incubation of the organic material and bacterial culture.

In various embodiments, mechanical degradation or chemical treatment of the organic material (e.g. feedstock) may be required at any point or multiple points in the system either to achieve an appropriate particle size or to render the carbonaceous components of the organic material more accessible to the respective digestion bacterial culture. Known methods of mechanical degradation may be used in accordance with the invention. Various pretreatment of the organic substrate can advantageously be used with the present invention, such as acid or alkaline hydrolysis.

Mechanical size reduction of organic material and feedstock has been found to aid with the biodegradation for several reasons. Physical size reductions corresponds to an increase in the active surface area of the particles to be digested. Mechanical size reduction may also rupture cell walls thereby making the biodegradable components more accessible to microorganisms. In various embodiments, the organic material is pretreated using a method comprising grinding the feedstock to a size from about 5 millimeters to about 50 millimeters. In various embodiments, the feedstock is heated to a temperature between about 50° C. and about 120° C., more preferably from about 60° C. to about 90° C. In various embodiments, the feedstock is pretreated by a physical process selected from the group consisting of grinding, cutting, heating and combinations thereof. The pretreatment may be upstream from the biogasification reactor.

In various embodiments, hydrolysis reactor 35 and biogasification reactor 46 contain bacterial culture to produce a biogas by biodigestion of an organic material including at least some solid components and biostabilization reactor 58 contains bacterial culture to produce a biogas by biodigestion of an organic material essentially free of solid components. “Essentially free” with respect to presence of solid components refers to less than about 10%, in various respects less than about 5%, in various respects less than about 3%, and in various respects less than about 1%. In various respects, an organic material “essentially free” of solid components refers to liquid waste.

The method of using the anaerobic digester system in accordance with the present invention will now be described. In various aspects, the system is operated similar to the digester systems disclosed by U.S. Pat. No. 7,556,737 (“the '737 patent”) and U.S. Pat. No. 6,342,378 (“the '378 patent”), the contents of which are hereby incorporated herein for all purposes by this reference. In various aspects, components of the system are operated similar to the systems disclosed by U.S. Pat. Nos. 4,316,961 and 4,722,741, the contents of which are hereby incorporated herein for all purposes by this reference.

Exemplary hydrolysis reactor 35 and biogasification reactor 46 are optionally heated and/or cooled intermittently and fed in sequential batches to promote energy conversion. Biostabilization reactor 65 may or may not be heated depending on various factors are understood from the description herein including, but not limited to, the climatic conditions and the specific content and distribution of the organic material. In various embodiments, hydrolysis reactor 35 and biogasification reactor 46 are insulated for heat conservation. In various embodiments, all the reactors—the hydrolysis reactor, the biogasification reactor, and the biostabilization reactor—are insulated for heat conservation.

In the exemplary method, organic materials with more than about 10% solids content is first fed into grinder 33 for mechanical size reduction. The resulting mixture includes solids in an aqueous solution. In the exemplary system, the solid particles have a diameter less than or equal to about 20 mm. In various embodiments, the size of the solid particles is continuously reduced in the grinder and the organic feedstock is continuously fed to the hydrolysis reactor.

Next the solid-containing matter serves as a feedstock for hydrolysis reactor 35. The feedstock is broken down by a combination of chemical and biochemical reactions in the hydrolysis reactor to produce a mixture of sugar, organic acids (e.g. amino acids and fatty acids) and alcohols (e.g. ethanol). Chemical hydrolysis takes place because of water, the hydrolytic and acidogenic bacterial culture, and enzymes present in the hydrolysis reactor. Biochemical reactions are carried out by the acidogenic and hydrolytic microorganisms.

The feedstock and contents of the hydrolysis reactor, including the hydrolytic and acidogenic bacterial culture constitute a hydrolysis mixture in the reactor. The hydrolysis mixture is retained in the reactor for a sufficient time and under sufficient conditions to produce a biogas. In the exemplary system, the biogas includes hydrogen and carbon dioxide as the main components and hydrogen sulfide and ammonia as the minor components. The biogas is removed through gas vent 44 and transferred to another location or stored.

After a period of incubation to allow the hydrolysis reactions to proceed up to an including completion in hydrolysis reactor 35, the hydrolysis effluent is transferred to biogasification reactor 46 via optional wet grinder 39 and pump 47. In the exemplary system, the hydrolysis effluent and acetogenic and methanogenic bacterial culture in the reactor form a biogasification mixture. In biogasification reactor 46, the sugars, organic acids, alcohols and other compounds in the biogasification mixture are converted into biogas by the acetogenic and methanogenic bacterial culture. The biogas produced from biogasification reactor 46 contains methane and carbon dioxide with hydrogen sulfide and ammonia being minor components. The biogas is removed through gas vent 53 and transferred to another location or stored. The biogas produced by the biogasification reactor may be mixed with the gas produced by the hydrolysis reactor, in which case the gas components are separated later. Alternatively, the biogas from each reactor may be kept separate.

After incubation for a period of time to allow most of biogasification to occur, the biogasification effluent is transferred to biostabilization reactor 58 through solid-liquid separator 54. In the exemplary method, the biogasification mixture is incubated for a sufficient period of time and under sufficient conditions to use up all or a portion of the solid components. Whereas existing anaerobic digester systems require incubating the organic material in the biogasification reactor until the contents are biostabilized, the system and method in accordance with the present invention provide for biostabilization in biostabilization reactor 58. The exemplary biogasification reactor will achieve about 80% to about 90% of the maximum biogas production potential from the biodegradable solid components.

In various embodiments, system 30 includes one or more processes for recycling processed liquid, solid, or a combination thereof. In various embodiments, at least part of the effluent from one or more of the reactors of the system is transferred to one or more reactors. All or part of the hydrolysis effluent may be transferred back into the hydrolysis reactor. All or part of the biogasification effluent may be transferred into the hydrolysis reactor. All or part of the biostabilization effluent may be transferred into the hydrolysis reactor, biogasification reactor, or a combination thereof. In various embodiments, the effluent of the respective reactors is not recycled.

In the exemplary system, the biogasification effluent is primarily a liquid with only minor, small solid particles after passing through optional solid-liquid separator 54. In exemplary system 30, part of this liquid is recycled to hydrolysis reactor 35. The recycled liquid may be fed into grinder 33 as an eluent, added to the hydrolysis reactor feedstock, and/or fed directly into the hydrolysis reactor. The recycled liquid from the hydrolysis reactor may be added to a feedstock mixing device such as a mixing tank or mixing pump prior to the hydrolysis reactor. The recycled liquid may replenish water and nutrients in the feedstock for the hydrolysis reactor.

The process of recycling the biogasification effluent in accordance with the invention is distinct from the recirculation process of U.S. Pat. No. 7,556,737 to Zhang. Unlike the recirculation of Zhang, which is continuous, the recycling process of the invention is performed in one or more batches as feedstock is added to the grinder. The recycling is performed, in part, to conserve liquid and reduce the use of municipal water. In the exemplary system, the effluent from biogasification reactor 46 is transferred to grinder 33 or pump 37 for adjusting the moisture content of the feedstock for hydrolysis reactor 35. In the exemplary system, the effluent is primarily liquid. The residual organic material, which includes organic acids, is converted to biogas in biostabilization reactor 58.

The biostabilization effluent may be used or treated in a conventional manner. For example, the biostabilization effluent may be further processed for water and nutrient recovery. The biostabilization effluent may also be used for crop irrigation. In various embodiments, the biostabilization is recycled to the hydrolysis reactor, biogasification, or both. The process for recycling the liquid component of the biostabilization effluent is similar to the process for recycling the biogasification effluent described above.

In the exemplary system, solids feedstock such as crop residues, rice straw, green waste, municipal waste, and the like are introduced to the hydrolysis reactor in batches or semibatches. Meanwhile, the biogasification reactor produces biogas substantially continuously. In various embodiments, the solids feedstock is fed into the hydrolysis reactor from the top of the reactor in batches or semibatches.

The system may include more than one hydrolysis reactor and other components as will be appreciated from the description herein. For example, the system may include a buffer tank. After the feedstock is hydrolyzed in multiple hydrolysis tanks, the effluent from the different hydrolysis tanks is collected and transferred to the buffer tank for equilibration. Hydrogen and carbon dioxide gases can also produced in the buffer tank. The equilibrated soluble substances are transferred intermittently to the biogasification reactor for continuous biogas production. After completing a digestion cycle, the digested straw is removed from the hydrolysis reactor before a new batch of straw is added. In various embodiments, the system includes more than one of the hydrolysis reactor, biogasification reactor, and biostabilization reactor.

One will appreciate from the description herein the manner for adjusting the incubating conditions for each of the hydrolysis reactor, biogasification reactor, and biostabilization reactor. In various embodiments, at least one of the temperature, pressure, and incubating time are maintained within a desired or predetermined range. In various embodiments, the system includes a controller and microprocessor for monitoring and controlling the conditions within one or more of the reactors and the flow rate.

In order to increase the reaction rates and efficiency in each reactor, the thermal and chemical conditions may be different. In various embodiments, the hydrolysis reactor is operated at a temperature between about 50° C. and about 55° C., the biogasification reactor is operated at a temperature between about 35° C. and about 40° C., and the biostabilization reactor is operated at a temperature between about 25° C. and about 30° C. In various embodiments, the hydrolysis reactor is operated at a temperature between about 35° C. and about 45° C., the biogasification reactor is operated at a temperature between about 35° C. and about 40° C., and the biostabilization reactor is operated at a temperature between about 25° C. and about 35° C.

In various embodiments, the hydrolysis reactor is operated at a pH between about 4.5 to about 6.5, the biogasification reactor is operated at a pH between about 6.8 and about 8.0, and the biostabilization reactor is operated at a pH between about 6.8 and 8.0. In various embodiments, the hydrolysis reactor, biogasification reactor, and biostabilization reactor are all operated at a pH between about 6.5 and about 8.2.

In various embodiments, the reduction in total solids (TS) achieved by the process is at least about 50%, preferably at least about 60% and more preferably at least about 90%. In various embodiments, the reduction in volatile solids (VS) is at least about 60%, more preferably at least about 70% and even more preferably at least about 80%. In various embodiments, the TS and VS reductions were, respectively, at least about 70% and at least about 80% for food waste, at least about 70% and at least about 80% for mixture of food and green wastes, and at least about 50% and at least about 70% for green waste. In various embodiments, the average biogas yield of the system and method of the invention is at least 300 mL/gVS, preferably at least 400 mL/gVS and still more preferably at least 500 mL/g/VS. In various embodiments, the system yields at least about 200 mL/gVS, preferably at least 300 mL/gVS and still more preferably at least 400 mL/g/VS. In another embodiment, the concentration in the hydrogen gas collected from the hydrolysis reactor is between about 10% to about 60%, and more preferably between about 20% to about 50%.

In various embodiments, the concentration of methane gas collected from the biogasification reactor is between about 40% to about 80%, more preferably between about 50% to about 70% and most preferably about 60%. In various embodiments, the concentration of methane gas collected from the biostabilization reactor is between about 60% to about 80%, more preferably between about 65% to about 80% and most preferably about 70%.

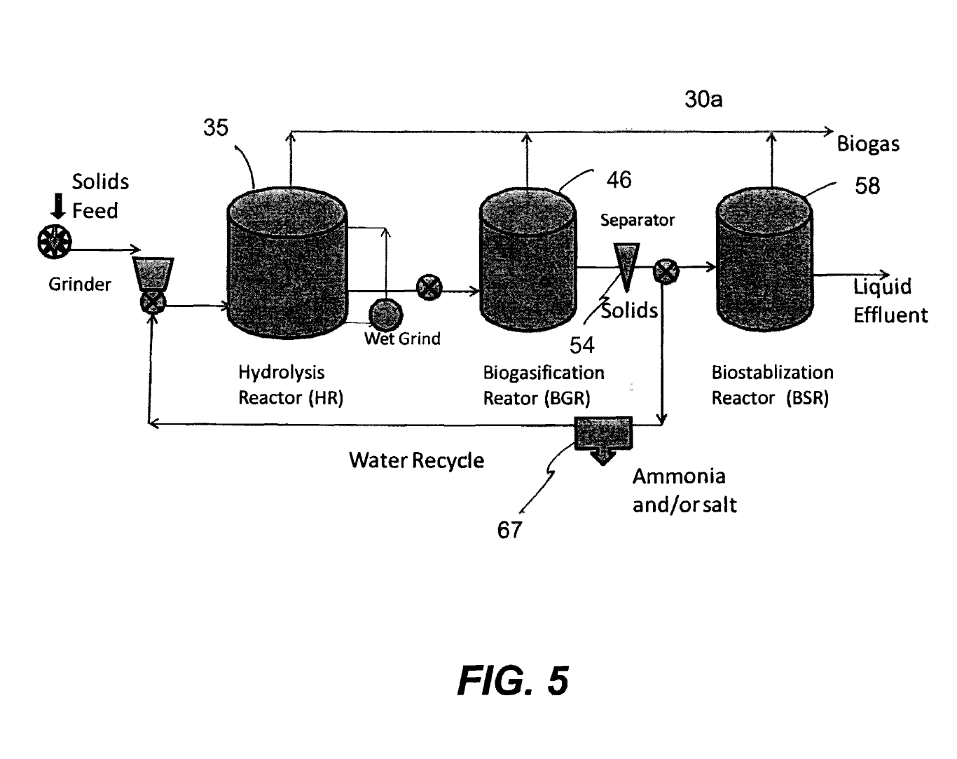

Turning to FIG. 5, an anaerobic digester system 30a in accordance with the present invention is shown. System 30a is similar to system 30 in many respects but includes an optional ammonia removal device 67. System 30a includes three anaerobic reactors: hydrolysis reactor 35, biogasification reactor 46, and biostabilization reactor 58.

Ammonia removal device 67 removes ammonia, salt, and other elements prior to recycling. The device 67 separates and removes the undesirable excess amounts of ammonia and salt. This may be necessary for treating organic material that has high protein content and salt, such as meat products.

The method of using the system 30a is similar to the method of using system 30. In various embodiments, the liquid separated by solid-liquid separator 54 is passed through ammonia removal device 67 to remove ammonia in the liquid prior to recycling to the hydrolysis reactor. The ammonia removal process can be a chemical, mechanical, or ionic process including, gas stripping, membrane separation, and other conventional techniques. In an exemplary embodiment, the liquid is treated with a base chemical (lime or sodium hydroxide) to increase pH above about 9, and at the same a gas (air or biogas) is passed through the liquid (e.g. bubbling) to strip ammonia from the liquid. Ammonia in the gas can be removed later from the gas and collected as ammonia product. One process for ammonia collection is to let the ammonia-laden gas react with acid (e.g. sulfuric acid or nitric acid). Ammonia will be reacted with acid to form ammonium sulfate or ammonium nitrate, which can be used as fertilizer products or for other purposes.

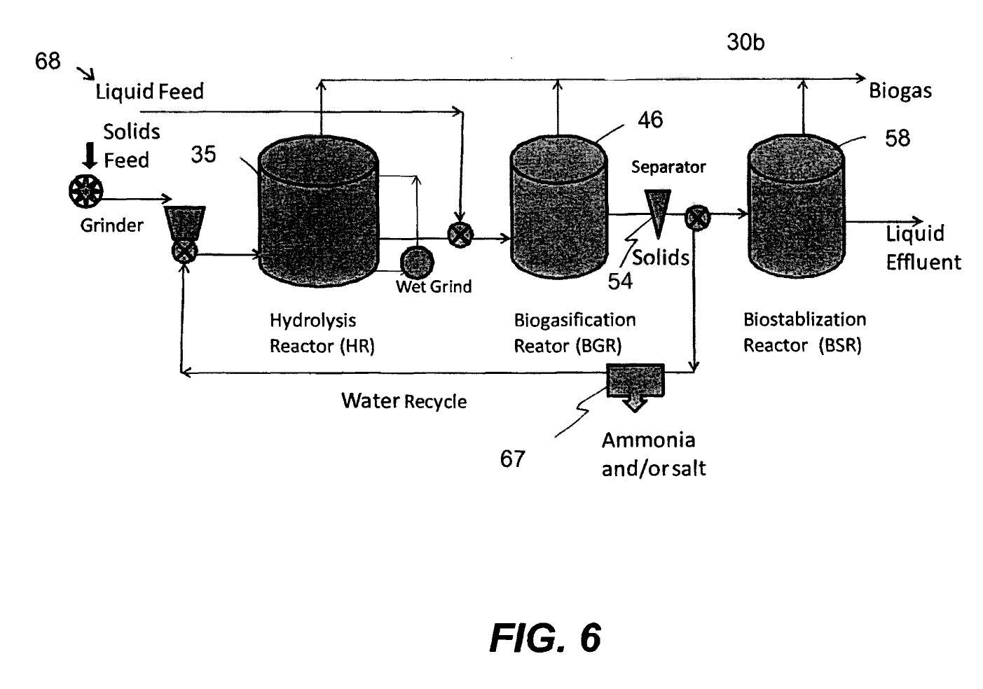

With reference to FIG. 6, an exemplary anaerobic digester system 30b in accordance with the present invention is shown. System 30b is similar to system 30a in many respects but except that biogasification reactor 46 receives fresh liquid feed 68 from an external source. In the exemplary system, the fresh liquid feed is wastewater. Liquid feed 68 is added to the biogasification reactor along with the effluent from hydrolysis reactor 35. System 30b is generally used in applications where both solid and liquid feedstock needs to be treated, for example, solid waste and wastewater.

In operation and use, system 30b is used in substantially the same manner as system 30a and system 30 discussed above.

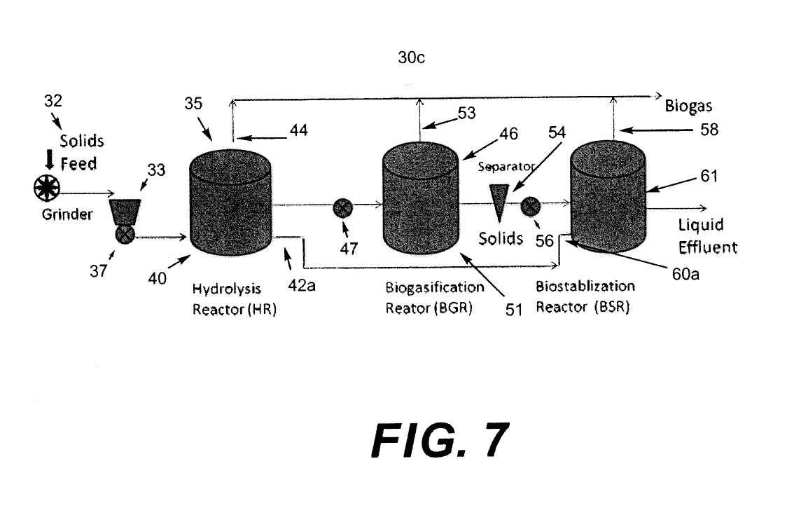

With reference to FIG. 7, an exemplary anaerobic digester system 30c further comprises a second hydrolysis effluent port 42a allowing the transfer of hydrolysis reactor effluent to biostabilization reactor 58 through second biostabilization inlet port 60a.

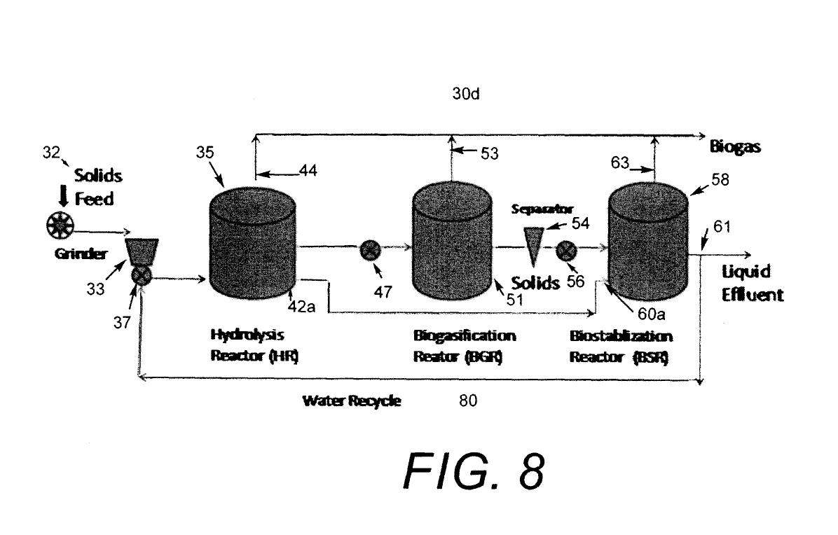

FIG. 8 shows an exemplary system of the invention 30d in which a hydrolysis reactor second effluent port 42a allows the transfer of hydrolysis reactor effluent to biostabilization reactor 58 through a biostabilization reactor second inlet port 60a. The system further comprises line 80, through which effluent from the biostabilization reactor is recycled back into the system, for example, into the hydrolysis reactor, for example, via valve 37.

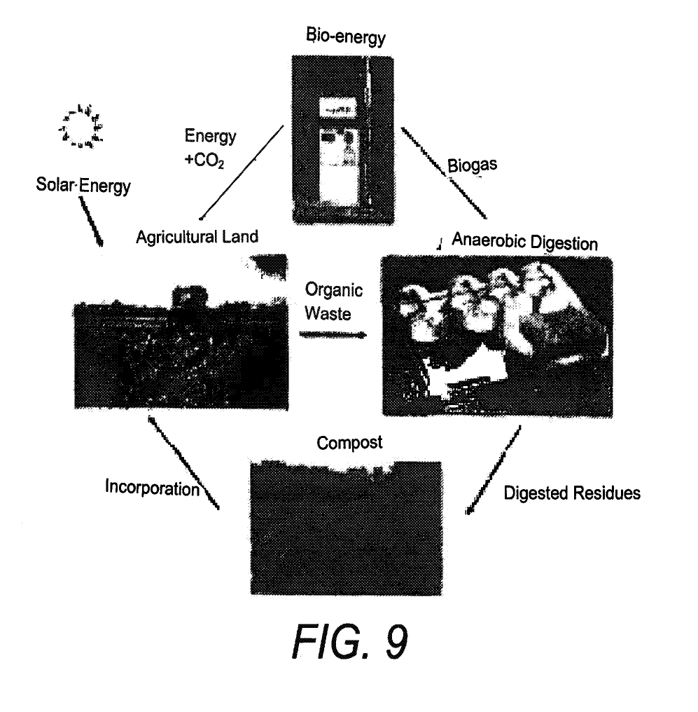

As one of the increasingly important technologies for biofuel production, anaerobic digestion provides many public benefits with regard to bioenergy generation, environmental quality protection, and public health improvement. FIG. 9 illustrates the many public benefits of anaerobic digestion and its byproducts.

By contrast to conventional high solid digesters, the high rate anaerobic biodigester system in accordance with the present invention provides increased energy efficiency for conversion of organic materials into biogas energy. The system of the invention can also be used to in more applications than any existing technologies. Through the use of the optional water recycling and ammonia and salt separation process, the system can be used to treat various organic solid materials with a wide range of chemical compositions.

The system and method in accordance with the present invention provide the capabilities and flexibilities to treat both solid waste and wastewater in one system. The system can be used for treatment of both solid waste and wastewater and production of biogas (e.g. hydrogen and methane gases) for energy generation. Consequently, the system may increase energy efficiency and lower the cost of the system.

In the exemplary system, biostabilization reactor 58 and biogasification reactor 46 are functionally and structurally different. In the exemplary system, solid components are digested by the biogasification reactor, separated by solid-liquid separator 54 for composting, or a combination thereof. Thus, the biostabilization reactor operates primarily on liquid waste. Because the organic material fed to the biogasification reactor and biostabilization are different, the bacterial culture contained in each reactor generally is different. In part for the above reasons, the biostabilization reactor allows for higher process rates and shorter retention time in comparison to systems with only a biogasification apparatus and process.

The system in accordance with the invention can reduce the organic content of waste and wastewater in comparison to existing systems.

Additionally, the exemplary system employs several features to make the biodigestion process more efficient and produces more biogas from a given organic material than existing anaerobic digestion systems. These optional features and benefits include at least (1) three biological and temperature phased anaerobic digestion processes to achieve optimum thermal, chemical and biochemical conditions for fast conversion of organic materials into biogas; (2) concurrent mechanical and biological breakdown of organic solids to enhance the rates of chemical and biochemical reactions; (3) water recycling to reduce the clean water usage and wastewater discharge; and (4) treatment of both solid waste and wastewater in one system.

The system in accordance with the invention can be used for producing biogas energy from organic materials, such as food and yard waste, agricultural residues, food processing byproducts, and animal manure.

The system in accordance with the invention provides more energy-efficient means than existing high solid digesters for conversion of organic materials into biogas energy. The system can be used to in more applications than any of the existing technologies. Because of the optional water recycling and ammonia and salt separation process incorporated in the digester system, the system can be used to treat various organic solid materials with a wide range of chemical composition.

Compared to existing anaerobic digestion systems, the anaerobic digestion system in accordance with the present invention has higher energy conversion efficiency at a lower cost, including capital, operational, and maintenance costs. Further, the system is easier to operate and maintain.

In summary, in various preferred embodiments, the present invention provides:

An anaerobic digester system for producing a biogas from organic material, said system comprising: a hydrolysis reactor comprising therein hydrolytic bacterial culture for which the organic material is a hydrolysis substrate, the hydrolysis reactor further comprising: a hydrolysis inlet port for receiving the organic material; a first hydrolysis outlet port for discharging hydrolysis effluent from the hydrolysis reactor; and a gas vent for discharging the biogas from the hydrolysis reactor; a biogasification reactor comprising therein acetogenic and methanogenic bacterial culture, the biogasification reactor further comprising: a biogasification reactor inlet port for receiving the hydrolysis effluent from the hydrolysis reactor outlet port; a biogasification reactor outlet port for discharging biogasification effluent from the biogasification reactor; and a gas vent for discharging the biogas from the biogasification reactor; and a biostabilization reactor comprising therein a methanogenic bacterial culture, the biostabilization reactor further comprising: a first biostabilization reactor inlet port for receiving the biogasification effluent from the biogasification reactor outlet port; a biostabilization reactor outlet port for discharging biostabilization effluent from the biostabilization reactor; and a gas vent for discharging the biogas from the biostabilization reactor.

An anaerobic digester system according to the preceding paragraph for producing a biogas from organic material, said system comprising: a hydrolysis reactor comprising therein a bacterial culture for producing a biogas from organic material comprising biodegradable solids, the hydrolysis reactor further comprising: a hydrolysis inlet port for receiving the organic material; a hydrolysis outlet port for discharging hydrolysis effluent from the hydrolysis reactor; and a gas vent for discharging the biogas from the hydrolysis reactor; a biogasification reactor comprising therein bacterial culture for producing biogas from organic material comprising biodegradable solids, the biogasification reactor further comprising: a biogasification reactor inlet port for receiving the hydrolysis effluent from the hydrolysis reactor outlet port; a biogasification reactor outlet port for discharging biogasification effluent from the biogasification reactor; and a gas vent for discharging the biogas from the biogasification reactor; and a biostabilization reactor comprising therein a bacterial culture for producing biogas from organic material essentially free of biodegradable solids, the biostabilization reactor further comprising: a biostabilization reactor inlet port for receiving the biogasification effluent from the biogasification reactor outlet port; a biostabilization reactor outlet port for discharging biostabilization effluent from the biostabilization reactor; and a gas vent for discharging the biogas from the biostabilization reactor.

A system according to any preceding paragraph, the biostabilization reactor including a vessel for holding the methanogenic bacterial culture, wherein the biostabilization reactor outlet port communicates with a vertical surface of the biostabilization reactor vessel.

A system according to any preceding paragraph, the biogasification reactor including a vessel for holding the methanogenic bacterial culture, wherein the biogasification reactor outlet port communicates with a vertical surface of the biogasification reactor vessel.

A system according to any preceding paragraph, wherein the biogasification reactor has a controlled internal temperature above about 30° C.

A system according to any preceding paragraph, wherein the biogasification reactor has a controlled internal temperature between about 25° C. and about 55° C.

A system according to any preceding paragraph, wherein the biogasification reactor has a controlled internal pH of between about 6.8 and about 8.2.

A system according to any preceding paragraph, wherein the organic material is a member selected from a solid, liquid, and a combination thereof.

A system according to any preceding paragraph, wherein the hydrolysis reactor further comprises acidogenic bacterial culture.

A system according to any preceding paragraph, wherein the biostabilization reactor has a controlled internal temperature equal to or below that of the biogasification reactor.

A system according to any preceding paragraph, wherein the biostabilization bacterial culture is essentially free of acetogenic bacteria.

A system according to any preceding paragraph, wherein the biostabilization reactor has a controlled internal pH of between about 6.8 and about 8.2.

The system according to any preceding paragraph, wherein the biogasification reactor is configured to process a member selected from a liquid, solid, and combination thereof.

A system according to any preceding paragraph, further comprising a grinder upstream from the biogasification reactor for mechanically reducing the size of solid particles in the organic material.

A system according to any preceding paragraph, further comprising: a solid-liquid separator positioned between the biogasification reactor and the biostabilization reactor, the separator configured to separate fibrous solid components from a liquid component of the biogasification effluent.

A system according to any preceding paragraph, wherein the fibrous solid component has moisture content between about 60% and about 70%.

A system according to any preceding paragraph, further comprising filter means fluidly positioned between the biogasification reactor and the biostabilization reactor.

A system according to any preceding paragraph, wherein the filter means is selected from one of a grinder, grid, filter, sieve, strainer, slats, and combinations thereof.

A system according to any preceding paragraph, wherein the biogas discharged from the hydrolysis reactor comprises hydrogen and carbon dioxide, the biogas discharged from the biogasification reactor comprises methane and carbon dioxide, and the biogas discharged from the biostabilization reactor comprises methane.

A system according to any preceding paragraph, wherein the organic material has a high salt content.

A system according to any preceding paragraph, further comprising a removal device for removing one of ammonia, salt, and a combination thereof from the biogasification effluent.

A system according to any preceding paragraph, further comprising a fluid line for transferring at least a portion of the biogasification effluent to the hydrolysis reactor via the removal device.

A system according to any preceding paragraph further comprising a biostabilization reactor second inlet port for receiving the biogasification effluent from a hydrolysis reactor second outlet port.

A system according to any preceding paragraph further comprising a biostabilization reactor effluent recycle line, feeding the biostabilization reactor effluent to a member selected from said hydrolysis reactor, said biogasification reactor and a combination thereof.

A method for producing a biogas comprising: delivering the organic material to the hydrolysis reactor of the system of any of the above claims as a feedstock; incubating a hydrolysis mixture comprising the hydrolysis effluent and the acidogenic and hydrolytic bacterial culture under anaerobic conditions to produce hydrogen, carbon dioxide, and the hydrolysis effluent; transferring at least a portion of the hydrolysis effluent to the biogasification reactor; incubating a biogasification mixture comprising the hydrolysis effluent and the acetogenic and methanogenic bacterial culture under anaerobic conditions to produce methane, carbon dioxide, and the biogasification effluent; transferring at least a portion of the biogasification effluent to the biostabilization reactor; and incubating a biostabilization mixture comprising the biogasification effluent and the biostabilization methanogenic bacterial culture under anaerobic conditions to produce methane and the biostabilization effluent. This method can, but does not have to be, practiced with any device or system set forth herein. In various embodiments, the method comprises transferring a portion of the effluent from the hydrolysis reactor to the biostabilization reactor.

A biostabilization reactor system for producing a biogas from a partially-digested organic material, said reactor system comprising: a vessel including an inlet for mixing the partially-digested organic material with a biostabilization bacterial culture for biogasification of the organic material; a gas vent for discharging biogas resulting from the biogasification; and an outlet port for discharging liquid effluent resulting from the biogasification from the vessel; wherein the partially-digested organic material has been submitted to methanogenesis with a mixture of acetogenic and methanogenic bacterial culture upstream from the vessel and the biostabilization bacterial culture is a methanogenic bacterial culture. The biostabilization reactor system can, but does not have to be, utilized in any device or system or in practicing any method set forth herein. The partially digested organic material is transferred to the biostabilization reactor from the biogasification reactor, the hydrolysis reactor or a combination of the two.

A system according to the preceding paragraph, wherein the methanogenic bacterial culture is essentially free of acetogenic bacteria.

A system according to any preceding paragraph, further comprising a solid-liquid separator for separating solid components from liquid components of the partially-digested organic material to be fed to the vessel.

A system according to any preceding paragraph, wherein the vessel is configured to maintain an internal temperature of between about 25° C. to about 55° C.

A system according to any preceding paragraph, wherein the vessel is configured to maintain the mixture of the organic material and the biostabilization bacterial culture at a pH of between about 6.8 and about 8.2.

A system according to any preceding paragraph, wherein the outlet port is configured to draw the liquid effluent from a region adjacent an inner wall surface of the vessel.

A system according to any preceding paragraph, wherein the discharged biogas is discharged from a top of the vessel.

A system according to any preceding paragraph, wherein said inlet is operably fluidically connected to a hydroylsis reactor such that hydrolysis reactor effluent is transferred into said system.

A method for producing a biogas which is a member selected from methane, hydrogen, carbon dioxide, and combinations thereof, said method comprising: delivering a feedstock, a portion of which comprises ground solid organic material, to a hydrolysis reactor, the hydrolysis reactor comprising hydrolytic and acetogenic bacterial culture for which the solid organic material is a hydrolysis substrate; incubating a hydrolysis mixture comprising the feedstock and the hydrolytic and acetogenic bacterial culture for a period of time and under sufficient anaerobic conditions to produce hydrogen, carbon dioxide, and a hydrolysis effluent; transferring a first portion of the hydrolysis effluent to a biogasification reactor comprising therein acetogenic and methanogenic biogasification bacterial culture; incubating a biogasification mixture comprising a second portion of said hydrolysis effluent and the biogasification bacterial culture for a period of time and under sufficient anaerobic conditions to produce methane, carbon dioxide, and a biogasification effluent; transferring at least a portion of the biogasification effluent to a biostabilization reactor comprising therein a biostabilization bacterial culture; and incubating a biostabilization mixture comprising the biogasification effluent and the biostabilization bacterial culture for a period of time and under sufficient anaerobic conditions to produce methane and carbon dioxide. The method also optionally further comprises transferring at least a portion of the hydrolysis effluent to the biostabilization reactor. The method may, but does not have to, be practiced with any device or system or as a component of or addition to any method set forth herein.

The method according to any preceding paragraph, wherein the biostabilization incubating is performed at a temperature equal to or lower than the biogasification incubation.

A method according to any preceding paragraph, further comprising providing a different liquid feedstock to the biogasification reactor prior to the biogasification incubating.

A method according to any preceding paragraph, further comprising, before the transferring to the biostabalization reactor, separating solid components from a liquid of the biogasification effluent.

A method according to any preceding paragraph, further comprising recycling a portion of the separated liquid to the hydrolysis reactor.

A method according to any preceding paragraph, wherein each of the steps is performed essentially simultaneously.

A method according to any preceding paragraph, wherein the biostabilization bacterial culture is a methanogenic bacterial culture essentially free of acetogenic bacteria.

A method according to any preceding paragraph, said method further comprising transferring at least a portion of the biogasification effluent to the biostabilization reactor.

### EXAMPLES

### Example 1

### High Rate Anaerobic Digester System Tested

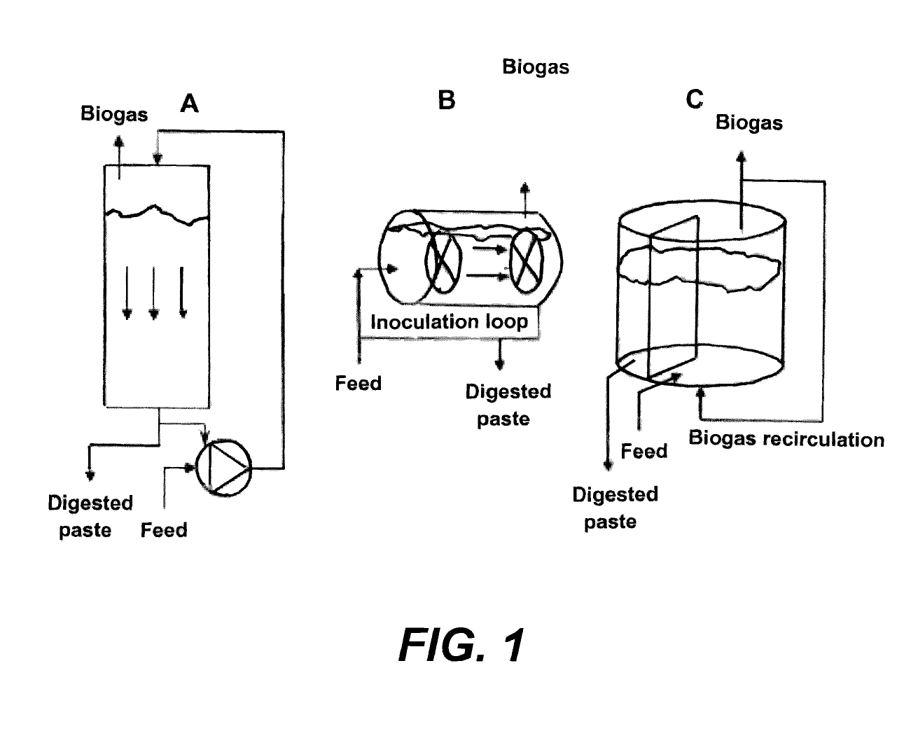

The High Rate Anaerobic Digester System (HR BioDigester) as shown in FIG. 1 was tested for treatment of vegetable waste. The HR BioDigester System had three reactors, hydrolysis reactor (HR), Biogasification reactor (BR) and Biostablization reactor (BSR). The HR, BR and BSR have working volumes of 5, 5 and 9 liters, respectively. All the reactors were operated at 35 degree C. The hydraulic retention time was 5 day for HR, 20 days for BR and 12 day for BSR. The mixture of three vegetables (including cabbage, green pepper and celery) was used as feedstock for the digester system and the HR BioDigester was tested for about 70 days. The vegetable mixture was prepared from the fresh vegetables using a laboratory food processor. The vegetable mixture had total solids (TS) and volatile solids (VS) contents of 6-7% and 5.5-6.5%, respectively, and consisted of 55% cabbage, 27% pepper, and 17% celery.

Vegetable mixture was first fed into the HR. In the HR, the vegetables were reacted by microorganisms, and became hydrolyzed and mostly converted into volatile fatty acids (acetic acid was the primary acid produced). The pH in the HR was maintained to be between 5 and 6 (5.6 to 5.8 mostly). The HR was intermittently mixed (3 min every hour) and then allowed to settle for two hours before effluent was drawn. The effluent of HR was drawn at two ports located on the wall of the reactor, one at approximately middle height (named upper port), and one close to the bottom (lower port). The effluent removed from the upper port contained less suspended solids than the effluent removed from the lower port. The effluent from the upper port was sent directly to the BSR and the effluent from the lower port was sent to BGR for converting into biogas. The effluent from the BGR was fed to the BSR for further treatment after passing through a solid-liquid separator (press) to remove part of solids. The effluent from BSR was discharged. The BGR and BSR were intermittently mixed (3 min every hour) and allowed to settle (with no mixing) for two hours before the effluent was discharged. The pH in the BGR and BSR was maintained in the range of 7.4-7.8.

Two tests were conducted. The first test was for system (a) in FIG. 1 and lasted for about 50 days with the first 30 days as system start up and the latter 20 days for system performance data collection. The second test was for system (b) in FIG. 1 and lasted for 30 days, following the first test. The first test was to feed the HR with only the vegetable mixture and the second test was to feed the HR with both the vegetable mixture and the recycled water taken from the BSR effluent. In the first test, ammonia hydroxide was added into the HR to increase the nitrogen content and alkalinity to control the pH. The second test was for recycling the nutrients in the system so that ammonia addition requirement was avoided. The amount of recycled water was the same as the amount of vegetable mixture. The vegetables and recycled water were mixed prior to being fed into the HR.

### Test Results

The biogas produced from the HR contained 5-30% hydrogen, 70-93% carbon dioxide and 2-4% methane. The biogas composition in the HR varied depending on the feeding conditions. The biogas produced from the BGR and BSR had stable composition with 70-72% methane and 30-28% carbon dioxide. In the first test, the average biogas yield from the digester system during the first test period was 624 ml/gVS, which was calculated based on the original vegetable mixture fed into the HR. The biogas yield was distributed among HR, BGR and BSR as 80, 116 and 428 ml/gVS, respectively. In the second test, the average biogas yield was 557 ml/gVS. The biogas yield distributed among HR, BGR and BSR was 76, 122 and 359 ml/gVS, respectively. The solids reduction achieved for both systems were 86-88% for total solids (TS) and 92-93% for volatile solids (VS). Because of the high digestability of vegetables, the solids removed from the BGR reactor effluent using the press was small, about 2% solids. There, over 85% of total solid and over 90% volatile solid was converted into biogas through microbial digestion processes.

### REFERENCES

CHYNOWETH, D. P., G. BOSCH, J. F. K. EARLE, R. LEGRAND and K. X. LIU (1991). “A novel process for anaerobic composting of municipal solid-waste.” Applied Biochemistry and Biotechnology 28-9: 421-432.
CHYNOWETH, D. P., J. OWENS, D. OKEEFE, J. F. K. EARLE, G. BOSCH and R. LEGRAND (1992). “Sequential batch anaerobic composting of the organic fraction of municipal solid waste.” Water Science and Technology 25(7): 327-339.
DE BAERE, L. (2000). “Anaerobic digestion of solid waste: state-of-the-art.” Water Science and Technology 41(3): 283-290.
DE BAERE, L. (2006). “Will anaerobic digestion of solid waste survive in the future?” Water Science and Technology 53(8): 187-194.
DE LACLOS, H. F., S. DESBOIS and C. SAINT-JOLY (1997). “Anaerobic digestion of municipal solid organic waste: Valorga full-scale plant in Tilburg, the Netherlands.” Water Science and Technology 36(6-7): 457-462.
EPA. (2008). “The AgSTAR Program: Guide to Anaerobic Digesters.” Retrieved May 20, 2008, from http://www.epa.gov/agstar/operational.html#addatabase.
FORSTER-CARNEIRO, T., L. A. FERNANDEZ, M. PEREZ, L. I. ROMERO and C. J. ALVAREZ (2004). “Optimization of SEBAC start up phase of municipal solid waste anaerobic digestion.” Chemical and Biochemical Engineering Quarterly 18(4): 429-439.
LISSENS, G., P. VANDEVIVERE, L. DE BAERE, E. M. BIEY and W. VERSTRAETE (2001). “Solid waste digesters: process performance and practice for municipal solid waste digestion.” Water Science and Technology 44(8): 91-102.
MARSH, M. L. H. and T. LAMENDOLA, California Energy Commission, PIER Program (2006). Dairy methane digester system 90-day evaluation report—Hilarides Dairy. CEC-500-2006-086.
NICHOLS, C. E. (2004). “Overview of anaerobic digestion technologies in Europe.” BioCycle 45(1): 47-53.
OKEEFE, D. M., D. P. CHYNOWETH, A. W. BARKDOLL, R. A. NORDSTEDT, J. M. OWENS and J. SIFONTES (1993). “Sequential batch anaerobic composting of municipal solid-waste (MSW) and yard waste.” Water Science and Technology 27(2): 77-86.
RAPPORT, J. L., R. ZHANG, R. B. WILLIAMS and B. M. JENKINS (2008). “Anaerobic digestion technologies for municipal waste treatment.” International Journal of Environment and Waste Treatment In Press.
TEN BRUMMELER, E. (2000). “Full scale experience with the BIOCEL process.” Water Science and Technology 41(3): 299-304.
TEN BRUMMELER, E., M. M. J. AARNINK and I. W. KOSTER (1992). “Dry anaerobic-digestion of solid organic waste in a BIOCEL reactor at pilot-plant scale.” Water Science and Technology 25(7): 301-310.
TEN BRUMMELER, E., H. HORBACH and I. W. KOSTER (1991). “Dry anaerobic batch digestion of the organic fraction of municipal solid-waste.” Journal of Chemical Technology and Biotechnology 50(2): 191-209.
VANDEVIVERE, P., L. DE BAERE and W. VERSTRAETE (2002). Types of anaerobic digesters for solid wastes. Biomethanization of the Organic Fraction of Municipal Solid Wastes. J. Mata-Alvarez. Barcelona, IWA Publishing: 111-140.
VERMA, S. (2002). Anaerobic digestion of biodegradable organics in municipal solid wastes, Columbia University. Master of Science.
ZHANG, R. H. and Z. Q. ZHANG (1999). “Biogasification of rice straw with an anaerobic-phased solids digester system.” Bioresource Technology 68(3): 235-245.
ZHANG, R. B. (2002). Biogasification of Organic Solid Wastes. Biocycle 43(1):56-59.
ZHANG, R. H., H. M. El-Mashed, K. Hartman, F. Wang, G. Liu, C. Choate, and P. Gamble. 2006. Characterization of food waste as feedstock for anaerobic digestion. Bioresource Technology 98(2007):929-935.
EL-MASHAD, H. M. and R. H. ZHANG. Co-digestion of food waste and dairy manure for biogas production. 2010. Bioresource Technology 101(2010):4021-4028.235-244.
ZHU, B., R. H. ZHANG, P. GIKAS, J. RAPPORT, B. M. JENKINS, and X. LI. 2010. Biogas production from municipal solid waste using integrated rotary drum and anaerobic phased solid digester system. Bioresource Technology 101(2010):6374-6380.

The above-referenced literature is incorporated herein for all purposes by this reference.

1. An anaerobic digester system for producing a biogas from organic material, said system comprising:
a hydrolysis reactor comprising therein hydrolytic bacterial culture for which the organic material is a hydrolysis substrate, the hydrolysis reactor further comprising:
a hydrolysis inlet port for receiving the organic material;
a first hydrolysis outlet port for discharging hydrolysis effluent from the hydrolysis reactor; and
a gas vent for discharging the biogas from the hydrolysis reactor;
a biogasification reactor comprising therein acetogenic and methanogenic bacterial culture, the biogasification reactor further comprising:
a biogasification reactor inlet port for receiving the hydrolysis effluent from the hydrolysis reactor outlet port;
a biogasification reactor outlet port for discharging biogasification effluent from the biogasification reactor; and
a gas vent for discharging the biogas from the biogasification reactor; and
a biostabilization reactor comprising therein a methanogenic bacterial culture, the biostabilization reactor further comprising:
a first biostabilization reactor inlet port for receiving the biogasification effluent from the biogasification reactor outlet port;
a biostabilization reactor outlet port for discharging biostabilization effluent from the biostabilization reactor; and
a gas vent for discharging the biogas from the biostabilization reactor.

2. An anaerobic digester system for producing a biogas from organic material, said system comprising:
a hydrolysis reactor comprising therein a bacterial culture for producing a biogas from organic material comprising biodegradable solids, the hydrolysis reactor further comprising:
a hydrolysis inlet port for receiving the organic material;
a hydrolysis outlet port for discharging hydrolysis effluent from the hydrolysis reactor; and
a gas vent for discharging the biogas from the hydrolysis reactor;
a biogasification reactor comprising therein bacterial culture for producing biogas from organic material comprising biodegradable solids, the biogasification reactor further comprising:
a biogasification reactor inlet port for receiving the hydrolysis effluent from the hydrolysis reactor outlet port;
a biogasification reactor outlet port for discharging biogasification effluent from the biogasification reactor; and
a gas vent for discharging the biogas from the biogasification reactor; and
a biostabilization reactor comprising therein a bacterial culture for producing biogas from organic material essentially free of biodegradable solids, the biostabilization reactor further comprising:
a biostabilization reactor inlet port for receiving the biogasification effluent from the biogasification reactor outlet port;
a biostabilization reactor outlet port for discharging biostabilization effluent from the biostabilization reactor; and
a gas vent for discharging the biogas from the biostabilization reactor.

3. The system of claim 1, the biostabilization reactor including a vessel for holding the methanogenic bacterial culture, wherein the biostabilization reactor outlet port communicates with a vertical surface of the biostabilization reactor vessel.

4. The system of claim 1, the biogasification reactor including a vessel for holding the methanogenic bacterial culture, wherein the biogasification reactor outlet port communicates with a vertical surface of the biogasification reactor vessel.

5. The system of claim 1, wherein the biogasification reactor has a controlled internal temperature above about 30° C.

6. The system of claim 1, wherein the biogasification reactor has a controlled internal temperature between about 25° C. and about 55° C.

7. The system of claim 1, wherein the biogasification reactor has a controlled internal pH of between about 6.8 and about 8.2.

8. The system of claim 1, wherein the organic material is a member selected from a solid, liquid, and a combination thereof.

9. The system of claim 1, wherein the hydrolysis reactor further comprises acidogenic bacterial culture.

10. The system of claim 1, wherein the biostabilization reactor has a controlled internal temperature equal to or below that of the biogasification reactor.

11. The system of claim 1, wherein the biostabilization bacterial culture is essentially free of acetogenic bacteria.

12. The system of claim 1, wherein the biostabilization reactor has a controlled internal pH of between about 6.8 and about 8.2.

13. The system of claim 1, wherein the biogasification reactor is configured to process a member selected from a liquid, solid, and combination thereof.

14. The system of claim 1, further comprising a grinder upstream from the biogasification reactor for mechanically reducing the size of solid particles in the organic material.

15. The system of claim 1, further comprising:
a solid-liquid separator positioned between the biogasification reactor and the biostabilization reactor, the separator configured to separate fibrous solid components from a liquid component of the biogasification effluent.

16. The system of claim 15, wherein the fibrous solid component has moisture content between about 60% and about 70%.

17. The system of claim 1, further comprising filter means fluidly positioned between the biogasification reactor and the biostabilization reactor.

18. The system of claim 17, wherein the filter means is selected from one of a grinder, grid, filter, sieve, strainer, slats, and combinations thereof.

19. The system of claim 1, wherein the biogas discharged from the hydrolysis reactor comprises hydrogen and carbon dioxide, the biogas discharged from the biogasification reactor comprises methane and carbon dioxide, and the biogas discharged from the biostabilization reactor comprises methane.

20. The system of claim 1, wherein the organic material has a high salt content.

21. The system of claim 1, further comprising a removal device for removing one of ammonia, salt, and a combination thereof from the biogasification effluent.

22. The system of claim 21, further comprising a fluid line for transferring at least a portion of the biogasification effluent to the hydrolysis reactor via the removal device.

23. The system of claim 1 further comprising a biostabilization reactor second inlet port for receiving the biogasification effluent from a hydrolysis reactor second outlet port.

24. The system of claim 1 further comprising a biostabilization reactor effluent recycle line, feeding the biostabilization reactor effluent to a member selected from said hydrolysis reactor, said biogasification reactor and a combination thereof.

25. A method for producing a biogas comprising:
delivering the organic material to the hydrolysis reactor of the system of any of the above claims as a feedstock;
incubating a hydrolysis mixture comprising the hydrolysis effluent and the acidogenic and hydrolytic bacterial culture under anaerobic conditions to produce hydrogen, carbon dioxide, and the hydrolysis effluent;
transferring at least a portion of the hydrolysis effluent to the biogasification reactor;
incubating a biogasification mixture comprising the hydrolysis effluent and the acetogenic and methanogenic bacterial culture under anaerobic conditions to produce methane, carbon dioxide, and the biogasification effluent;
transferring at least a portion of the biogasification effluent to the biostabilization reactor; and
incubating a biostabilization mixture comprising the biogasification effluent and the biostabilization methanogenic bacterial culture under anaerobic conditions to produce methane and the biostabilization effluent.

26. A biostabilization reactor system for producing a biogas from a partially-digested organic material, said reactor system comprising:
a vessel including an inlet for mixing the partially-digested organic material with a biostabilization bacterial culture for biogasification of the organic material;
a gas vent for discharging biogas resulting from the biogasification; and
an outlet port for discharging liquid effluent resulting from the biogasification from the vessel;
wherein the partially-digested organic material has been submitted to methanogenesis with a mixture of acetogenic and methanogenic bacterial culture upstream from the vessel and the biostabilization bacterial culture is a methanogenic bacterial culture.

27. The system of claim 26, wherein the methanogenic bacterial culture is essentially free of acetogenic bacteria.

28. The system of claim 26, further comprising a solid-liquid separator for separating solid components from liquid components of the partially-digested organic material to be fed to the vessel.

29. The system of claim 26, wherein the vessel is configured to maintain an internal temperature of between about 25° C. to about 55° C.

30. The system of claim 26, wherein the vessel is configured to maintain the mixture of the organic material and the biostabilization bacterial culture at a pH of between about 6.8 and about 8.2.

31. The system of claim 26, wherein the outlet port is configured to draw the liquid effluent from a region adjacent an inner wall surface of the vessel.

32. The system of claim 26, wherein the discharged biogas is discharged from a top of the vessel.

33. The system of claim 26, wherein said inlet is operably fluidically connected to a hydroylsis reactor such that hydrolysis reactor effluent is transferred into said system.

34. A method for producing a biogas which is a member selected from methane, hydrogen, carbon dioxide, and combinations thereof, said method comprising:
delivering a feedstock, a portion of which comprises ground solid organic material, to a hydrolysis reactor, the hydrolysis reactor comprising hydrolytic and acetogenic bacterial culture for which the solid organic material is a hydrolysis substrate;
incubating a hydrolysis mixture comprising the feedstock and the hydrolytic and acetogenic bacterial culture for a period of time and under sufficient anaerobic conditions to produce hydrogen, carbon dioxide, and a hydrolysis effluent;
transferring a first portion of the hydrolysis effluent to a biogasification reactor comprising therein acetogenic and methanogenic biogasification bacterial culture;
incubating a biogasification mixture comprising a second portion of said hydrolysis effluent and the biogasification bacterial culture for a period of time and under sufficient anaerobic conditions to produce methane, carbon dioxide, and a biogasification effluent;
transferring at least a portion of the biogasification effluent to a biostabilization reactor comprising therein a biostabilization bacterial culture; and
incubating a biostabilization mixture comprising the biogasification effluent and the biostabilization bacterial culture for a period of time and under sufficient anaerobic conditions to produce methane and carbon dioxide.

35. The method of claim 34, wherein the biostabilization incubating is performed at a temperature equal to or lower than the biogasification incubation.

36. The method of claim 34, further comprising providing a different liquid feedstock to the biogasification reactor prior to the biogasification incubating.

37. The method of claim 34, further comprising, before the transferring to the biostabalization reactor, separating solid components from a liquid of the biogasification effluent.

38. The method of claim 37, further comprising recycling a portion of the separated liquid to the hydrolysis reactor.

39. The method of claim 34, wherein each of the steps is performed essentially simultaneously.

40. The method of claim 34, wherein the biostabilization bacterial culture is a methanogenic bacterial culture essentially free of acetogenic bacteria.

41. The method according to claim 34, said method further comprising transferring at least a portion of the hydrolysis effluent to the biostabilization reactor.
